(19) United States
(12) Patent Application Publication
(10) Pub. No.: US 2026/0248844 A1
(43) Pub. Date: Aug. 27, 2026

(54) COMPOSITIONS AND METHODS TO DISINFECT, TREAT AND PREVENT MICROBIAL INFECTIONS

(71) Applicant: WIAB WATER INNOVATION AB, Malmo (SE)

(72) Inventors: Pål Rongved, Oslo (NO); Aina Kristin Pham, Fornebu (NO); Thomas Bjarnsholt, Oslo (NO)

(21) Appl. No.: 19/540,110

(22) Filed: Feb. 13, 2026

Related U.S. Application Data
(63) Continuation of application No. 17/404,941, filed on Aug. 17, 2021.
Application No. 17/404,941 is a continuation-in-part of application No. 17/369,620, filed on Jul. 7, 2021.
(60) Provisional application No. 63/048,815, filed on Jul. 7, 2020.

(51) Int. Cl. A61K 33/20; A61P 31/14

(52) U.S. Cl. CPC A61K 33/20; A61P 31/14

(57) ABSTRACT

The present invention provides stable antimicrobial and disinfectant compositions comprising use of a solid precursor of an oxidized state of chlorine. The invention also provides on-demand storage and mixing vessels and methods for preparing and delivering on demand formulations. In addition, the invention provides antiviral, antibiotic and general antimicrobial uses, in vivo, on surfaces and via spray applications.

### CROSS-REFERENCE TO RELATED APPLICATION(S)

This application is a continuation-in-part of U.S. application Ser. No. 17/369,620, filed Jul. 7, 2021, which claims priority to, and the benefit of, U.S. Provisional Application No. 63/048,815, filed on Jul. 7, 2020, the content of each of which is incorporated by reference herein in its entirety.

### TECHNICAL FIELD

The present invention relates generally to new compositions comprising combinations of a solid or liquid precursor of an oxidized state of chlorine and acetic acid or its salts, wherein such compositions are useful disinfectants for treating a broad spectrum of bacterial and/or viral, fungal and parasitic pathogens, and collectively denoted microorganisms herein.

### BACKGROUND

Infectious diseases are a leading cause of death worldwide and account for more than 13 million deaths annually including nearly two-thirds of all childhood mortality. Moreover, antibiotic resistance is increasing and is contributing to morbidity in a broad range of human diseases, including pneumonia, tuberculosis and cholera. Of particular concern is that a number of human pathogen have developed resistance to conventional antibiotics. The introduction of new, more potent, derivatives of existing antibiotics only provides a temporary solution, since existing resistance mechanisms rapidly adapt to accommodate the new derivatives. Although resistant Gram-positive bacteria pose a significant threat, the emergence of multidrug resistant (MDR) strains of common Gram-negative pathogens, such as Escherichia coli, are of particular concern. In addition, isolates of Pseudomonas aeruginosa, Acinetobacter baumannii and Enterobacteriaceae have been shown to be resistant to virtually all antibiotics.

Viruses are also a significant concern in infectious epidemiology. Serious viral outbreaks, many of zoonotic origin, are becoming increasingly common. For example, the SARS (severe acute respiratory syndrome) and MERS (Middle East respiratory syndrome) outbreaks in the early-to-mid 2000s, the H1N1 pandemic in 2009, and the subsequent SARS COV-2 pandemic in 2020 have focused attention on both treatment and prevention of the spread of these viral pathogens.

Many viruses that infect the respiratory tract are communicated via droplet infection. In that case, respiratory droplets containing virus are expelled by an infected person and picked up by others on direct contact or by contact with surfaces on which the droplets land. Typically, infection proceeds via the binding of the virus to receptors on mucosal or epithelial cells, as a result of entry into the nose, eyes, ears, or mouth. In addition, some viruses are transmitted via aerosol particles containing the virus or are air borne. In either case, the virus may survive for hours to days after expression from an infected individual.

Conventional compositions and methods for disinfection of surfaces or contaminated epithelia are not sufficient for the inactivation of all infectious agents. Current forms of conventional disinfectant compositions may require long and impractical exposure times, or may use hazardous or corrosive solutions or vapors that cannot be used on sensitive instruments or on living tissues, and thus fail to provide practical solutions to growing health risks from resistant pathogens.

Chlorine oxides, or oxidized chlorine (also referred to herein as “OC”), comprise a large class of chemical species, and are often found in nature, as well as biological systems in mammals. Chlorine oxides may also exist as neutral compounds or ions, so-called oxyanions. There are several oxyanions of chlorine, in which an oxyanion can assume oxidation states of +1, +3, +5, or +7 with the corresponding anions hypochlorite (ClO⁻), chlorite (ClO₂⁻), chlorate (ClO₃⁻), or perchlorate (ClO₄⁻). The standard reduction potentials at a low pH of hypochlorous acid (HOCl) is +1.63, and for chlorous acid (HClO₂), the standard reduction potential is 1.64, while at basic pH, it is +0.89 and +0.78 respectively. At a pH of 5 to 7, reduction potentials are higher than +1.

Consequently, hypochlorite and chlorite are generally the most useful oxidation states with a potential to kill microbes and parasites. In particular, the chloride ion Cl⁻ is in the most stable oxidation state and is not reactive, nor is it effective as a disinfectant. Chlorate and perchlorate in oxidation states +5, and +7 are more reactive than the lower oxidation states, and may be more difficult to handle.

The hypochlorite ion has the chemical formula ClO⁻, where chlorine (Cl) is in oxidation state +1, which is a potentially unstable oxidation state since the low-energetic oxidation state of Cl is −1. Both the hypochlorite ion and the chlorite ion combine with a number of cations to form hypochlorites and chlorites, as the salts of these oxidized chlorines. Common examples include sodium hypochlorite (household bleach) and calcium hypochlorite, the main active ingredient of commercial products including bleaching powder, chlorine powder, or chlorinated lime, generally used for water treatment (e.g., swimming pools and the like). The chlorite and hypochlorite ions also referred to herein as the “main chlorine oxides”, are useful in various contexts. Sodium chlorite and hypochlorite are strong oxidizing agents, and have been used in water purification, disinfection, as well as bleaching and deodorizing animal products.

Because sodium hypochlorite produces a highly toxic chlorine gas under acidic conditions, commercially available aqueous solutions for household purposes are strongly basic solutions, with the pH adjusted using sodium hydroxide.

Hypochlorous acid is a weak acid that is known to rapidly inactivate bacteria, algae, fungus, and other organics, making it an effective agent across a broad range of microorganisms. Additionally, hypochlorous acid is generally non-toxic to humans because it is a weak acid and people naturally produce certain compounds that allow them to tolerate hypochlorous acid. Due to the combination of its biocidal properties and its safety profile, hypochlorous acid has been found to have many beneficial uses across many different industries, such as the medical, food service, food retail, agricultural, wound care, laboratory, hospitality, dental, or floral industries.

Hypochlorous acid is formed when chlorine dissolves in water. In particular, the acidification of hypochlorite generates hypochlorous acid, where the chlorine atom is in oxidation state +1. Hypochlorous acid exists in equilibrium with chlorine gas, which can escape from solution. The equilibrium is pH-dependent, as illustrated in the following equation (Equation 1):

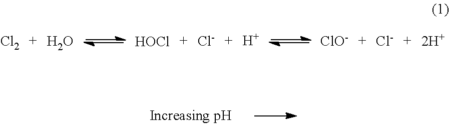

With reference to the above equation (Equation 1), a high pH drives the reaction to the right, promoting the disproportionation of chlorine into chloride and hypochlorite, whereas a low pH drives the reaction to the left, promoting the release of chlorine gas (Cl₂), which can be toxic.

A significant challenge with prior medical uses of solutions of chlorine, especially in higher oxidation states than −1, is its stability, since these chemical species are in a higher energy state and tend to return to the chloride ion Cl⁻ and will decompose in solution at ambient temperature. This prohibits the required shelf life stability at ambient conditions of pharmaceutical formulations and medical devices of chlorine oxides. Accordingly, a proper shelf life, as required for medical devices and drugs, is difficult to achieve for solutions of chlorine oxides. This inherent limitation to all oxides of chlorine restricts transport and storage, especially at higher temperature in areas with variable temperature, light humidity, and atmospheric gases.

Thus, while formulations containing chlorine oxides can be effective antimicrobial agents, conventional formulations have significant drawbacks. For example, the weak acid HOCI is unstable and impure when produced under conventional conditions. Consequently, there is a need for a more controlled, and immediate preparation processes that can furnish chlorine oxides on site with a stability that permits the intended short-term use. In general, there a significant unmet medical need for new therapeutics to treat resistant microbials and viruses.

### SUMMARY

The present invention provides disinfectant compositions comprising precursors of oxidized states of chlorine dissolved in a pharmaceutically acceptable diluents, adjuvants, or carriers and combined with activators. The resulting compositions provide improved antimicrobials for use in vivo, as well as for surface disinfection. In a preferred formulation, compositions of the invention comprise an acetic acid activator in combination with a form of hypochlorite. Optionally, formulations of the invention may be combined with a viscosity enhancer, and/or a dye. For example, the viscosity of formulations of the invention can be adjusted to form a gel using viscosity enhancers. Formulations of the invention are preferably mixed in a container comprising separate chambers as part of a multi-compartment device prior to use. Compositions of the invention may be formulated for oral, intravenous, dermal, or inhalation-based administration. In addition, formulations of the invention can be prepared for inhalation via a nebulizer or similar device for rapid introduction to a patient's respiratory system. As such, compositions of the invention are useful disinfectants for treating a broad spectrum of bacterial and/or viral pathogens, both in vivo and on surfaces.

In a particular aspect, the present invention is directed to antimicrobial formulations that provide a safe and effective means of treating and preventing respiratory infections, including both viral and bacterial infections. A preferred composition comprises a hypochlorous acid-based broad-spectrum antiviral and/or antibacterial inhalation solution. Solutions of the invention are preferably nebulized for inhalation delivery. More specifically, a preferred formulation comprises hypochlorous acid (HOCl) (from about 25 ppm to about 200 ppm) that is stabilized with acetic acid (approximately 0.25%), resulting in sustainable concentrations of HOCl with significant antimicrobial effects. The addition of acetic acid increases HOCl stability, thus making it possible to develop a treatment with extended shelf-life. Furthermore, the composition preferably is formulated at pH 5.5 and is physiologically isotonic thereby to increase tolerability within airways.

Compositions of the present invention have unique anti-pathogenic properties. In one aspect, compositions of the invention act on enveloped viruses, and provides superior antiviral effects against Corona-type viruses. Accordingly, such compositions are particularly useful for the treatment, and preventing the spread, of SARS infections (e.g., COVID-19). More specifically, SARS-COV-2 and many other viruses have surface proteins (i.e., spike proteins), which are entry points into cells of the respiratory system. These spike proteins comprise —SH groups vulnerable to oxidation by HOCl. Even relatively low concentrations of HOCl oxidizes extracellular —SH groups (e.g., on viral spike proteins), while being harmless to normal tissue and intracellular enzymes. As such, the antiviral effect of compositions of the present invention destroy viral particles in the respiratory tract upon first exposure, during infection, and when virions are intracellular and subsequently released by cells in the respiratory tract.

Therefore, the unique virucidal properties of compositions of the present invention, especially on enveloped viruses, makes such compositions a powerful tool in ongoing efforts to prevent the spread of coronaviruses. Such compositions reduce the duration of disease and severity of symptoms amongst a broad population of patients,

In another aspect, the present invention provides a disinfectant composition which includes a solid oxidized chlorine species salt, an activator, such as acetic acid, and a pharmaceutically-acceptable diluent, adjuvant, or carrier. The solid oxidized chlorine species salt is based on the formula Mⁿ⁺[Cl(O)ₓ]ₙⁿ⁻, where M is an alkali metal, alkaline earth metal, or transition metal ion, n is 1 or 2, and x is an integer between 1 and 4, inclusive. The activator is based on the formula R₁XOₙ(R₂)ₘ, where the R₁ group comprises between 1 and 10 hydrogenated carbon atoms, optionally substituted with amino, amido, carboxylic, sulfonic or hydroxy groups, wherein group X is selected from carbon, phosphorous and sulfur; n and m are each independently 2 or 3, and R₂ is selected from H, an alkali metal, an alkaline earth metal, a transition metal ion salt, and an ammonium salt.

In preferred embodiments, the oxidized chlorine salt comprises an alkali metal or alkaline earth metal salt of hypochlorous acid HOCl. In such an embodiment, the activator is acetic acid. In other embodiments, the oxidized chlorine salt comprises an alkali metal or alkaline earth metal salt of chlorous acid HOClO. Again, in such an embodiment, the activator is acetic acid.

In some embodiments, the composition comprises an osmolality in the range of about 0.1 mOsm to about 500 mOsm.

In some embodiments, an amount of oxidized chlorine species salt, acetic acid or its metal or ammonium salt produces a pH between 4 and 8.

In some embodiments, the composition further includes a viscosity-enhancing agent. In some aspects, the viscosity-enhancing agent cannot be oxidized by the oxidized chlorine species.

In some embodiments, the viscosity-enhancing agent comprises a water-soluble gelling agent. The water-soluble gelling agent may include, but is not limited to, poly acrylic acid, polyethylene glycol, poly(acrylic acid)-acrylamidoalkylpropane sulfonic acid co-polymer, phosphino polycarboxylic acid, and poly(acrylic acid)-acrylamidoalkylpropane, and sulfonic acid-sulfonated styrene terpolymers.

In some embodiments, the composition comprises a dye. The dye preferably produces a colorimetric indicator of the presence an oxidized chlorine compound in the formulation. The dye may be a reduction-oxidation dye. In a preferred embodiment, the color and intensity of the dye is dependent on the oxidation state of the oxidized chlorine compound.

Formulations of the invention may be composed as an aqueous solution, gel, cream, ointment, or oil. Formulations of the invention may be produced and stored in a multi-compartment container. In some aspects, aqueous and solid components are contained within separate respective compartments prior to combination.

Formulations of the invention are useful as antimicrobials on surfaces as well as for application to disease treatments. As such formulations of the invention are useful as inhalation products for use with, for example, a nebulizer, inhaler, vaporizer or other suitable means of delivery. In In addition, compositions of the invention can be formulated for application to skin, wounds mastitis or any other infectious diseases in animal or agricultural breeding; as well as antiviral applications.

### BRIEF DESCRIPTION OF THE DRAWINGS

FIG. 1 is a schematic illustration showing an exemplary multi-compartment or multi-chambered container for producing, storing, and dispensing a disinfectant composition according to embodiments of the present invention.

FIG. 2 shows the results obtained using sample solutions according to the present invention.

FIG. 3 shows the results obtained using sample solutions according to the present invention.

### DETAILED DESCRIPTION

The present invention relates generally to compositions comprising a combination of a solid and liquid precursor of an oxidized state of chlorine and an activator, e.g. acetic acid or its salts, as well as one or more additional components. The use of such compositions acts as disinfectants for treatment of a broad spectrum of bacterial and/or viral pathogens on a variety of biotic and abiotic surfaces and environments.

Some preferred formulations of the invention are in a solid form, multi-component (i.e., two-component, three-component, four-component, etc.) formulation that instantaneously generates compositions with long-term stability. This reduces limitations related to shelf life typically observed with conventional solutions of hypochlorous acid or chlorine dioxide described in the prior art. More specifically, the immediate generation of ready to use formulations of the oxidized chlorine species from solid precursors (API-P) may be performed in a multi-compartment device or container at the site of use. The multi-compartment device or container is used for the preparation, dispensing, and long term, stable storage of prepared compositions consistent with the present invention. In particular, such multi-compartment containers described herein may have a number of compartments or chambers separately containing the components required to produce compositions of the present invention. In one example, the formulation comprises a solid precursor of an oxidized state of chlorine and acetic acid or its salts, a viscosity enhancer, and a dye) and is subsequently combined to prepare the antimicrobial composition at the desired time of use and on site.

Another chlorine oxide useful as an API in antimicrobial formulations is chlorine dioxide, wherein the chlorine atom is in oxidation state +3. The main reaction of sodium chlorite is the generation of chlorine dioxide, as illustrated in the following equation (Equation 2):

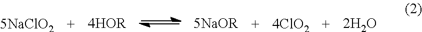

Referring to the above equation (Equation 2), HOR is usually a mineral acid, such as HCl or citric acid, since a source of protons is needed to convert sodium chlorite, first to chlorous acid, and then to chlorine dioxide, which is a highly water-soluble gas at room temperature.

An advantage of chlorine dioxide is that it cannot generate chlorine gas, Cl₂ which is known to react to chlorinated hydrocarbons, e.g. trihalo-methanes, which are toxic environmental pollutants. Another advantage of chlorine dioxide is that the activity as a disinfection agent or stability of its water solutions is not pH-dependent.

The present invention addresses challenges associated with prior art compositions using chlorine oxides. In particular, the present invention provides compositions comprising a combination of solid precursors of oxidized states of chlorine (OC) and activators providing a source of protons. A preferred example of an activator is acetic acid or its salts, wherein the disinfectant compositions of the invention are instantly formed in a controlled and immediate process at the site of use with a stability that permits the intended short-term use. Such compositions are useful disinfectants for treatment of a broad spectrum of microorganisms. In particular, when the active pharmaceutical ingredient is generated from stable, solid precursors, referred to hereinafter as “API-P” of chlorine oxides at the site of use, the inclusion of e.g. acetic acid as an activator that simultaneously is buffering the solution or gel to a biocompatible pH value, the stability issue in prior art is no longer present.

As previously described, the technical solutions in the prior art fail to address how to secure an ionic strength or osmolality of the final antimicrobial solution biocompatible with biological fluids. Even further, the prior art fails to show how to regulate and increase contact time and persistence of the API in a region of therapeutic interest, e.g. by regulating rheology and fluidity. Yet still, the prior art fails to provide a relatively simple, yet effective, means of monitoring an oxidation state of the API and visual indication of where the API has been applied during mixing of a disinfectant composition.

Additionally, in some embodiments, compositions of the present invention may further include the use of a viscosity enhancer (also referred to herein as “VE”) and/or include a combination of a solid precursor of an oxidized state of chlorine and the activator, e.g. acetic acid or its salts.

Another embodiment of the invention is the inclusion of a dye in the formulation, preferably e.g., a redox sensitive dye, with a color that varies with the oxidation state of the chlorine atom.

In particular, some preferred compositions of the invention are in a solid form, multi-component (i.e., two-component, three-component, four-component, etc.) formulation, separated by breakable walls or barriers that instantly generates the composition with long-term stability. This eliminates any issues related to shelf life seen with solutions of hypochlorous acid or chlorine dioxide described in the prior art.

More specifically, the immediate generation of ready-to-use formulations of the oxidized chlorine species from solid precursors API-P may be performed in a multi-compartment device or container at the site of use. The multi-compartment device or container may be used for the preparation, dispensing, and long term, stable storage of prepared compositions consistent with the present invention. In particular, such multi-compartment containers described herein may have a number of compartments or chambers separately containing the components required to produce the compositions of the present invention. In example, the solid precursor of an oxidized state of chlorine and the activator, e.g. acetic acid or its salts, a viscosity enhancer, and a dye is mixed, and subsequently the composition generates at the desired formulation of the disinfectant at the desired time and site of use.

Acetic acid is an abundant natural compound found various mammalian tissues. It is also a by-product of bacterial fermentation of carbohydrates.

Sodium acetate is non-toxic and is allowed in drug formulations for oral and parental use. The bactericidal effect of acetic acid is well known. It has a documented effect against problematic Gram-negative bacteria such as P. vulgaris, P. aeruginosa and A. Baumannii and others. The microbiological spectrum of acetic acid is wide, even when tested at a low concentrations of 0.5-3%. The concentrations of acetic acid that eradicated a pre-formed biofilm ranged from 0.10% to 2.5. Thus, acetic acid and its metal salt are very attractive compounds to use in antimicrobial formulations because of its ability to act as a buffer together with its metal salt for stabilization of pH.

Further, in addition to its antimicrobial properties, acetic acid is attractive because it cannot be oxidized further by oxidizing agents, such as an OC, and because of its endogenous nature in high concentrations in living tissue.

Accordingly, the multi-compartment container enables practical use in mixing the components necessary to generate the active solution of the API instantly and at the site of use. It should be noted that, to secure an ionic strength or osmolality of the final antimicrobial solution to adapt to the osmolality on the region of use in the case of medical applications, a pre-calculated amount of NaCl can be included in the multi-compartment device, dependent on the planned use.

A preferred embodiment of the invention is an inhalation formulation for respiratory administration. Thus, nebulizers or inhalators, generally used for the treatment of cystic fibrosis, asthma, COPD and other respiratory diseases or disorders, that convert liquids into aerosols are useful in the present invention. A device for inhalation administration may use compressed air or ultrasonic energy to generate atomization of the formulations of the invention. pressurized metered dose inhalers (pMDIs), dry powder inhalers (DPIs), slow mist inhalers (SMIs) of any kind, are also useful. Any electrostatic or non-electrostatic inhalators, e.g. the VORTEX or Pari or Sympotec are also useful to practice the invention.

The pre-loaded multi-compartment container described herein produces a stable, broad-spectrum antimicrobial solution upon mixing of the components, and leaves only biocompatible inactive chemical species in nature.

As noted above, the activation of the API of the invention is produced using an activator, e.g. acetic acid, which acts synergistically with oxidized chlorine against microbes, and further maintains acidity in pH range between 4 and 8. The inventive method and the formulations thereof avoids the inherent lack of long-term stability of oxidized chlorine OC in solution, since there is no need to store the disinfectant composition as a water solution.

Another advantage of the present invention is the option to add other compounds that will aid in application. For example, in wound healing applications, there is a need to increase the viscosity (μ) of the product on the skin to prolong contact time. The invention solves for this problem by the use of a water-soluble or dissolvable viscosity enhancer (VE) that chemically cannot be oxidized by the API, thereby providing improved regulation of contact time and persistence of the API in a region of therapeutic interest. The VE ensures that the rheology and fluidity is adapted to the respective method and region of disinfection, to generate a solution with full fluidity or a gel. The VE may include, for example, a water-soluble gelling agent such as polyacrylic acid, polyethylene glycol or any other oligomer or polymer that cannot be oxidized by the API.

Additionally, compositions of the invention may include a one or more dyes, preferably selected from a group of reduction-oxidation dyes (also referred to herein as “ROD” or “RODs”), wherein the color and intensity is dependent on the oxidation state of oxidized chlorine. It should be noted that, in addition to providing a visual indication (i.e., by way of color) of the oxidation state of the chlorine atom, the RODs further provide an antimicrobial effect of their own. This enhances the synergistic action between the components in the formulation in a novel way. The ROD is able to maintain its color for a period of time sufficient to monitor the oxidative activity of the API, oxidized chlorine, and further provide a visual indication of the region wherein the formulation has been applied.

Additional advantages of the invention, as well as additional inventive features, will be apparent from the description of the invention provided hereinafter.

In a preferred embodiment, the oxidized chlorine species (OC), has the general formula denoted below:

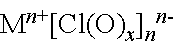

wherein M is any alkali metal, alkaline earth metal or transition metal ion, n is an integer 1-5, and x is an integer 1-4

If M=Na, n=1, x=1, the API-P is the solid NaOCl. If M=Ca, n=2, x=1, the API-P is the solid Ca(OCl)₂. If M=Na, n=1, x=2, the API-P is the solid NaClO₂. If M=Ca, n=2, x=2, the API-P is the solid Ca(ClO₂)₂. In the case in which x=3 or 4, the API-P generates the more reactive chlorate and perchlorate species.

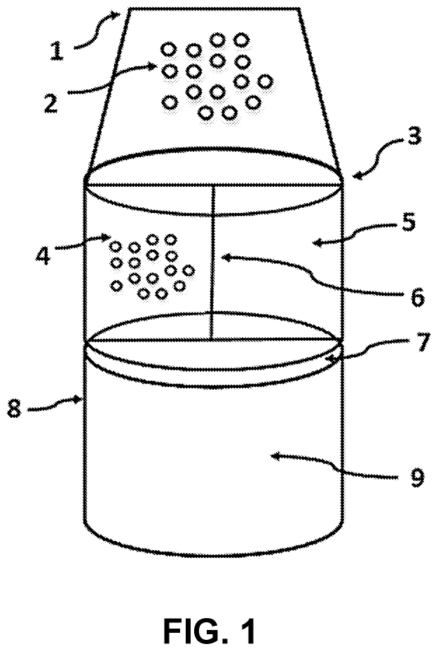

One non-limiting example is instant generation of hypochlorous acid from sodium-, or calcium-hypochlorite in the cap 2 according to FIG. 1, with a solution of sodium acetate buffer in compartment 4 providing a ready-to-use solution of the API hypochlorous acid with a pH between 5 and 6 in compartment 9, optionally with a color and a viscosity enhancer.

Another non-limiting example is calcium di-hypochlorite Ca(OCl)₂, which is a stable and water-soluble API-P for HOCl. It is instantly soluble in water, and only leaves calcium hydroxide, which is present in nature, and which generates HOCl, one of the two the active ingredient in the present invention, which degrades to Cl⁻ and biocompatible species containing hydrogen and oxygen.

Another preferred embodiment of the present invention is a solid precursor of oxidized chlorine is tetrachloro-decaoxide (TCDO), CAS no. 92047-76-2, known as WF10 or stabilized solutions of OXO-K993, prepared as described by Meuer et al in CA2616008, incorporated by reference herein. It can be prepared by combining alkaline or alkaline earth salts of the chlorite ion ClO₂⁻ with excess oxygen in water.

Thus, one advantage of the present invention is that the solid form precursors API-P in a dry and water-free quality is devoid of pharmaceutical stability issues, thus the present invention solves one of the main technical problems in prior art.

An aspect of the invention is the combination of the API-P with a molecule comprising a carboxylic acid functionality —COOH, a sulfonic acid functionality —SO₃H, a phosphoric acid functionality —PO₃H or a boric acid functionality —B(OH)₂, each of which serves as the activator of the API-P in the formulation. In general, the activator has the general formula R₁XOₙ(R₂,)ₘ wherein the group R₁ may be a group comprising from about 1 to about 10 hydrogenated carbon atoms, optionally substituted with amino, amido, carboxylic or hydroxy groups. The group X may be a carbon, phosphorous or sulfur atom, n and m is 2 or 3 and R₂ is a proton (H), or any alkali metal, alkaline earth metal or transition metal ion. The nature of the substituents in the formula varies according to use and chlorine species, and may be any compound comprising an amino group, e.g. ammonia, an amino acid, e.g. taurine or a therapeutic drug increasing the synergistic potential of the formulation. The activator may be any combination or mixture of two or more compounds as defined by the general formula R₁XOₙR₂.

Preferred non-limiting examples are carboxylic acids R₃COOH, wherein R₃ is H, or a linear or branched saturated or unsaturated hydrocarbon chain with from about 1 to about 24 carbon atoms, optionally substituted with hydroxyl groups. Non-limiting examples of activator may be acetic acid, citric acid, tartaric acid, lactic acid, hippuric acid, maleic acid, boric acid, sulfuric acid, phosphoric acid, boric acid, 3-(N-morpholino) propanesulfonic acid (MOPS), 2-(carbamoylmethylamino) ethanesulfonic acid (ACES), 2-(carbamoylmethylamino) ethanesulfonic acid (ADA), 2-(carbamoylmethylamino) ethanesulfonic acid (bicine), piperazine-N,N′-bis(2-ethanesulfonic acid, PIPES), or any amino acid.

Taurine is especially preferred, since it is the endogenous amino acid normally moderating the effect of OC in the body, and may be combined with OC to form endogenous N-chloro-amino acids like ClNH—CH₂CH₂—SO₃H, which in itself has antibacterial properties.

Acetic acid is preferred because it is endogenous in humans, has antibacterial properties, has very low toxicity and forms buffers in admixture with is metal salts, and is used as a non-limiting example in the further description of the invention.

An advantage of the invention is that the solid multi-component products according to the invention is not hampered by stability issues in a pharmaceutical or medical device setting, regardless of temperature, air, humidity, light, oxygen or other ambient conditions, since the API-Ps are solid and commercially available in large scale.

The API-Ps disclosed herein can be soluble in water, and nearly instantly reach physiological pH and ionic strength in the final solution in combination with acetic acid and/or its salts.

The ability to instantaneously generate the antimicrobial API in situ increases the ease of use and versatility of the product. In addition, the packaging of the components can be separate and combined on demand, further impacting storage stability and use in the field.

Small, stable single-use two or three-component devices are contemplated by the invention, ideally suited for travel, catastrophic response, military personnel, or microbial pandemics. Further, design of large formats (e.g., tanks) containing the precursors of the active antimicrobial (sometimes referred to herein as API-P) is useful in agricultural settings, aquaculture industry or military operations, and is suitable to disinfect larger areas.

### Viscosity Enhancers for Preparations of Viscous Solutions and Gels

In some embodiments of present invention components other than the API can be included. For example, a viscosity enhancer is preferred for wound healing or skin disinfection. Preferred viscosity enhancers are water-soluble gelling agents that do not oxidize the API. The gelling agents provide prolonged persistence of the API at the area of interest, e.g., skin.

Examples of gelling agents according to the invention include, but are not limited to, poly acrylic acid (CARBOMER), polyethylene glycol or any other oligomer, polymer or block-copolymer thereof. Further, the viscosity enhancer may be selected from, poly(acrylic acid)-acrylamidoalkylpropane sulfonic acid co-polymers, phosphino polycarboxylic acids and poly(acrylic acid)-acrylamidoalkylpropane and sulfonic acid-sulfonated styrene terpolymers.

Polymers, such an acrylate copolymer, function well in formulations of the invention in concentration ranges from about 0.01 to about 5%. Acrylate copolymers are homo- and co-polymers of acrylic acid cross-linked with a polyalkenyl polyether. Acrylate copolymers exist in a variety of graft densities. One exemplary cross-linker is pentaerytritol, which is very stable. Polyacrylic acid (PAA) polymers which are known to stabilize formulations of H₂O₂, can be used with the present invention.

The polymer-stabilized solutions of OC according to the invention have applications in many contexts, e.g. in wound treatment, aseptic packaging, electronics manufacture, and pulp and paper bleaching. The API can be formulated as a gel or viscous fluid, which may be applied to target surfaces, either inanimate or representative of the infected epithelial mucosal or skin surfaces so as to ensure prolonged and intimate contact with the necessary levels of API. Non-viscous formulations of the API may also be dispersed into the air in confined spaces as a mist in order to achieve environmental disinfection, or for inhalation purposes for treatment of respiratory diseases. For example, a concentration of the poly acrylic acid CARBOMER has increasing viscosity in the concentration 0.01-0.1%. If desired, it forms regular gels in the concentration range 0.1-1%.

### Antibacterial Redox-Sensitive Antibacterial Dyes as Indicators

A further additive to formulations of the invention is reduction-oxidation dyes (hereinafter ROD), wherein the color and intensity of the dye is dependent on the oxidation state of the OC. Even more advantageous, RODs themselves have antimicrobial effects, increasing the antimicrobial synergy between constituents of the formulations presented herein. If the standard half-cell potential of the ROD has a lower positive value than OC, the color of the formulation will be maintained as long as the OC is active. Thereby, the color provides a visual clue in the region wherein the formulation has been applied and where there is active OC. This is especially advantageous when, for example, a formulation according to the invention is used in treatment of mastitis, where large packs of cattle needs to be treated for mastitis; the colored formulation according to the invention visualized which animals have been treated. Further, employment of the opposite type of indicator, where the color appears when the oxidizing power of the OC is vanishing, is also useful.

Non-limiting examples of suitable dyes useful in the invention, are pH-independent dyes, visible in the presence of an OC. Preferred examples are N-phenylanthranilic acid (violet-red), N-ethoxychrysoidine (cyan), o-dianisidine (red), sodium diphenylamine sulfonate (red-violet), diphenylbenzidine (violet), diphenylamine (violet) and viologen, which is colorless in the presence of an OC, but deep blue in the absence of an OC.

Examples of pH-dependent dyes that are deep blue in the presence of an active OC, but colorless in the absence of the OD are sodium 2,6-Dibromophenol-indophenol or Sodium 2,6-Dichlorophenol-indophenol, sodium o-cresol indophenol, thionine (syn. Lauth's violet), methylene blue, Gentian Violet, indigotetrasulfonic acid, indigo carmine (syn. Indigo-disulfonic acid), indigomono sulfonic acid. Examples of dyes that are red or red-violet in the presence of an OC are phenosafranine, Safranin T, neutral red and dialkyl-p-phenylenediamine (SPD, red violet).

Many of these dyes have antibacterial effects in their selves, i.e. methylene blue (MB) and Gentian Violet (GV), and combinations of them have been used as antibacterial dyes in foams in wound dressings in combination with polymers like polyvinyl alcohol or polyurethane, e.g. as described by Edwards in Advances in Wound Care (2016), 5, pp 11-19.

A particularly useful class of dyes useful in the present invention is microbial phenazines, which are pigmented, redox-active, nitrogenous aromatic compounds with metabolic, ecological and evolutionary significance.

An additional class of phenazines include the bis-N-oxide phenazines, with even stronger antimicrobial properties than their parent phenazines. Most of these compounds are natural compounds produced by bacteria, and are hetero-aromatic N-oxidized compounds, hereinafter denoted HANOX. In addition to being redox dyes, RODS, the HANOX compounds are useful in the present invention because their color is dependent on the oxidation state of the OC.

Further, certain phenazine derivatives. In particular, they demonstrated a high activity against a wide variety of bacterial, yeasts and fungi such as Streptococcus agalactiae, Staphylococcus aureus, Escherichia coli, Corynebacterium pyogenes, Moraxella bovis, Pseudomonas aeruginosa, Candida albicans and Microsporum canis. Thus, the phenazine derivatives are particularly useful in the treatment of animal diseases of microbial origin in agriculture.

A surprising finding with these derivatives is their lack of injurious effects to tissue under the conditions of use, making them particularly suitable for topical application, preferably employed in amount ranging from 0.05 percent to 1.0 percent by weight of the composition.

They are of particular value in topical applications, e.g. in solid or gel formulations including finely divided powders and granular materials and in liquid formulations including solutions, suspensions, concentrations, tinctures, slurries and aerosols, creams, gels, jellies, ointments and pastes.

Methylene blue is another particularly preferred dye useful in the invention, since FDA has approved it as an excipient in drug formulations and it has antibacterial properties and its effects as a therapeutic agents can be enhanced using photodynamic therapy.

### Multi-Compartment Devices Useful in the Invention

FIG. 1 is a schematic illustration showing an exemplary multi-compartment device for instant generation of a formulation according to the invention.

The design of the device, including the number of compartments, can be adapted to the specifications of use. The device 8 consists of a screw cap 1 associated with the primary compartment, containing the solid precursor of the API, denoted API-P in a dry form (2). The screw cap 1 has the ability to open the seal or port 3 by turning it in one direction, letting the API-P into the second compartment 4, comprising a water solution of the activator also comprising a pre-calculated amount of sodium chloride to render the final osmolality of the solution to be iso-osmolal with body fluids. To gain the desired final pH, the activator and optionally a pre-calculated amount of its metal or amino acid salt in water may optionally be pre-loaded into compartment 4, 5 or 9. The smaller grains in 4 illustrate that the API-P is rapidly dissolving in the activator solution to generate the API. The third compartment 5 is optional and may contain a solution of a redox dye (ROD), depending on the technical use of the respective device. Compartments 4 and 5 are separated by a wall 6. Compartments 4 and 5 are also separated from compartment 9 by a breakable septum or wall 7. Optionally, a fourth compartment at the same level as 4 and 5 can contain an amino acid, e.g. an essential or non-essential amino acid or taurine for stabilization of the API. For simplicity, in the present illustration, the fourth compartment 9 may optionally be pure water, the activator solution. When turning the screw cap 1 in the opposite direction, the ready-to-use disinfectant solution is released from the device and may be applied at the region of interest for disinfection. An aspect of the invention is the solid precursor API-P in the screw cap 1, which is the oxidized chlorine species. The resulting solution from the multi-compartment device may eventually be used to produce a solution of the viscosity enhancer (VE), in a water solution. Any multi-chamber device that functions to permit mixing of the precursor components and additives is useful in the context of the invention, including bottles, bags, syringes, inhalators, hand disinfection devices, spay bottles, flasks, or tanks. As noted above, devices that can be easily activated bedside or in the field without complicated mixing procedures and can be stored at ambient temperatures are preferred.

The multi-compartment device according to the invention is a closed system and may be designed to eliminate mixing errors, to avoid undesired exposure to patients and personnel, and meets the Joint Commission and USO 797 guidelines.

Non-limiting examples of design useful in the invention are the Duplex Container from B Braun, the Credence Companion Safety Syringe System, the Dual-Mix multi-chamber bags or the Easyrec kit comprising a screw cap releasing a solid or mixture of solids for mixing into one or more fluid phases to generate the ready to use formulation of the API.

Use of the Invention for Antimicrobial Purposes with Photodynamic Therapy

Bacterial elimination using antimicrobial photodynamic therapy (aPDT) has been shown using the alternative therapeutic modality in peri-implantitis treatment. Thus, another preferred embodiment of the present formulation comprising an OC, acetic acid or its salt, optionally a viscosity enhancer, is the inclusion of a ROD exemplified by methylene blue for the use of photodynamic therapy, e.g. to improve wound healing or bacterial infections in mammals. In this case, the site of administration of the product according to the present invention can be irradiated with light with a wavelength adapted to generation of the photodynamic effect of the dye.

In Photodiagnosis Photodyn. Ther. (2018), 23, pp 347-352, Souza et al used photodynamic therapy to show antimicrobial activity of hypochlorite solutions and reciprocating instrumentation associated with photodynamic therapy on root canals infected with enterococcus faecalis. However, the test solutions was devoid of an antibacterial dye. These technologies are included in the present invention by reference.

### Stepwise Method for API Production

The present invention provides compositions and methods of the use of solid precursors API-P of chlorinated species, combined with the activator, e.g. acetic acid or its salt, and methods of its use. An exemplary method comprises the following 6 steps:
1. A pre-calculated amount of API-P having the general formula denoted below Mⁿ⁺[Cl(O)ₓ]ₙⁿ⁻, wherein M can be any alkali metal, alkaline earth metal or transition metal ion, wherein n is an integer 1-5, x is an integer 1-4, y is an integer 1-2. The solid state (API-P) generates a concentration of the API in the final solution in the form of an OC in the interval 0.01-1000 ppm, preferably in the range 0.1-100 ppm, is loaded into compartment 1 of a multi-compartment device. The API-P is mixed with a precalculated amount of NaCl to generate a final osmolality in the interval 0.1-500 mOsm, and optionally any other stabilizing solid.
2. A precalculated amount of an activator with the general formula R₁XOₙ(R₂,)ₘ, wherein the activator is preferably acetic acid, optionally in a mixture with its metal or ammonium salt. The activator is dissolved in a pharmaceutically acceptable diluent, adjuvant, or carrier to generate a concentration of the activator in the interval 0.05-10%, preferably in the range 0.08 to 0.5%, even more preferably in the range 0.10-0.2%. If the API-P is not premixed with NaCl, the solution in step 2 may comprise an amount of NaCl from step, either way generating a final osmolality in the interval 0.1-500 mOsm, preferably around 300 mOsm, corresponding to 150 mM NaCl. An aliquot of the solution is loaded into a second compartment of a multi-compartment device.
3. To generate the main product according to the invention, compartment 1 and 2 are mixed by opening a port or breaking a seal, membrane barrier or between the first and second compartments to mix the contents in the compartments, followed by ambient squeezing or shaking to generate the disinfectant solution. The resulting solutions can be taken out through a cap on the multi-compartment device prior to use. The solution is isotonic, has a pH in the interval 4 to 9, preferably between 5 and 6, and is generally used for antimicrobial purposes, e.g. for inhalation therapy using e.g. an asthma inhaler or nebulizer to fight viral infections in the upper airways in mammals.
4. For applications where a color indicator in step 4 can add information in the therapeutic procedure, e.g. in treatment of mastitis, or for indication of the oxidative activity of the API, a dye with a color that varies with the oxidation state of the API (ROD), in a precalculated amount to generate a concentration of the dye in the concentration range 0.01-1000 ppm, is optionally loaded into an optional compartment of a multi-compartment device.
5. Depending on the intended use, a precalculated amount an amino acid as a stabilizer of the API, preferably taurine in the same concentration as the API, is optionally is optionally loaded into an optional compartment of a multi-compartment device. Step 4 is performed to reduce oxidative stress to biological surfaces.
6. Depending on the intended use, an amount of a water-soluble viscosity enhancer (VE) that cannot be oxidized by the API in the concentration range 0.01-25%, preferably in the range 0.1-10%, even more preferably in the range 0.2-1%, is mixed with the solution resulting from a selected sequence of steps 1-3, optionally combined with any of the steps step 4-5. A VE concentration of 0.01-0.1% generates a viscous but fluid solution, while 0.3-1% produce a gel. For skin or wound applications, the third compartment in step 3. comprising the VE can be included in the mixing procedure to produce a viscous or gel-formed product.

### Use of the Invention in Agriculture

In agriculture, especially in animal farms, many kinds of infectious diseases caused by bacteria, viruses and fungi affect the daily operation of the farm, and affects the costs in running the facilities. In these settings, designed formulations according to the invention act therapeutically or prophylactically, and are especially useful in skin infections.

One important example is mastitis in cattle, which costs the US dairy industry about 1.7-2 billion USD each year. Effective and environmentally friendly treatment of mastitis has proven difficult, since milk from cows, having received long-term antibiotics is not marketable until the residual drugs have left the system. No vaccines are effective, since the infection in the udder and teats of the cow is remote from the animal's main blood stream. To mark cows having received treatment, dairy workers apply strips of tape to alert and mark treated cattle.

Thus, a preferred aspect of the present invention is treatment of mastitis using a gel or viscous solution comprising an OC, acetic acid or its salt, the viscosity enhancer VE and a ROD exemplified by methylene blue. The colored gel stays on the area of the udder and teats, acetic acid has the ability to penetrate into the skin of the teats, and the color makes use of strips of tape unnecessary. Additionally, the applied gel can be irradiated using light with suitable wavelength to increase the therapeutic effect of the gel. In this case, steps 1-4 and step 6 is performed to yield the instant formulation of use.

### Use of the Invention in Aquaculture

Water quality is a prerequisite for a successful culture of aquatic animals, exemplified by fish, oysters, prawns and shrimps. Open water systems often bring organisms like virus, bacteria, lice, protozoa, fungal pathogens, algae and parasites. Common virus infections that lead to high mortality in aquatic species attractive for food production are Koi Herpes Virus Disease, Pancreas disease (PD) and infectious salmon anemia (ISA). Proper water quality or sufficient quantity of pure water is most often not available. The breeding installations in prior art often has no means of hindering these infectious species to approach and effect the breeding species. Further, once infected, there is no efficient cure to provide efficient therapy against these diseases.

A preferred embodiment of the oxidized chlorine species OC according to the present invention is effective treatment of all these infections and harmful organisms and cells. The instant formulations of the OC are highly effective in controlling these waterborne pathogens. In example, chlorine dioxide is a broad-spectrum biocide effective to solve the defined problems in the prior art. The formulations in the present invention is even employed in special tanks to repeatedly treat e.g. bred salmon without harming the fish gills or any other parts of the bred species, while having a destructive effect on the microorganisms causing the disease. In these applications, the preparations sequence wherein the API-P is NaOClO₂ or Ca(OClO₂)₂ is loaded into compartment 1 and mixed with a precalculated amount of acetic acid in step 1-3 is used.

### Antiviral Use of the Invention

Methods disclosed herein for treatment of contaminated surfaces, equipment, e.g. medical equipment, furniture surfaces, doorknobs, devices, clothing or personnel. Formulations of the invention can be applied as a gel, aqueous solution, or by misting or vaporization of the API into a surface or confined space. The fact that the formulation is prepared on demand makes it possible to treat areas with high-potency antimicrobial without concern for storage degradation.

Methods of the invention contemplate dispersion of the active agents into crevices and microenvironments, even onto personnel who are suspected of having been contaminated by infectious tissues or bodily fluids. Vaporization of these formulations may enable beneficial therapeutic or prophylactic impacts on resistant viral, bacterial or fungal infections.

Formulations of the invention can be applied without substantial toxicity risk. A preferred embodiment is the treatment of a viral infection in the upper airways. Thus, systems and methods of the invention provide oxidized chlorine, OC, as a means of treating viral infection in the respiratory tract. Compositions of the invention are useful for treating SARS, MERS and other infections, including but not limited to, SARS COV-2 infections. This has now for the first time been facilitated through the instant precursors of the API combined with the multi-compartment device according to the invention, since there is no need to evaluate the lack of activity of a solution that has been stored at ambient conditions.

Particularly, inhalable hypochlorous acid formulations of OC, an activator, e.g. acetic acid, an excipient regulating the rheology of the final solution, an osmolality-regulating agent, e.g. sodium chloride. Such instant formulations can now be prepared on site, along with methods of delivery via a nebulizer, such as soft mist inhalers, jet nebulizers, ultrasonic wave nebulizers, and vibrating mesh nebulizers may be used. Upon use, inhalers and nebulizers aerosolize compositions of the invention for delivery via inhalation.

Formulations for aerosolization may be provided in dry powder form, solution, or suspension form. Fine droplets, sprays, and aerosols can be delivered by an intranasal or intrapulmonary pump dispenser or squeeze bottle. Compositions can also be inhaled via an inhaler, such as a metered dose inhaler or a dry powder inhaler. Compositions can also be inhaled via a nebulizer, such an ultrasonic wave nebulizer, providing compositions of OC and acetic acid directly to respiratory tracts via inhalable formulations. This prevents and treats infections of the respiratory system caused by viruses as well as other microbes. According to the invention, formulations as described herein are safe and effective for the prevention and treatment of viral infections.

Compositions of the invention may also include a pharmaceutically acceptable carrier, such as a diluent, to facilitate delivery to the respiratory mucosa. The carrier might be an aqueous carrier such as saline. The composition may be isotonic, having the same osmotic pressure as blood and lacrimal fluid. Suitable non-toxic pharmaceutically acceptable carriers are known to those skilled in the art. Various carriers may be particularly suited to different formulations of the composition, for example whether it is to be used as drops or as a spray, a suspension, or another form for pulmonary delivery.

Formulations for inhalation may be provided in dry powder form, solution, or suspension form. The composition can be delivered by various devices known in the art for administering drops, droplets, and sprays. The composition can be delivered by a dropper, pipet, or dispenser. Fine droplets, sprays, and aerosols can be delivered by an intranasal or intrapulmonary pump dispenser or squeeze bottle.

Intranasal delivery may be provided via a nasal spray device. Accordingly, the formulations according to the invention may be designed as a nasal spray. The nasal spray is insufflated into the nose and is delivered to the respiratory tract.

Soft mist inhalers use mechanical energy stored in a spring by user-actuation to pressurize a liquid container, causing the contained-liquid to spray out of a nozzle for inhalation in the form of a soft mist. Soft mist inhalers do not rely on gas propellant or electrical power for operation. The average droplet size in soft mist inhalers is about 5.8 micrometers.

Jet nebulizers are the most commonly used and may be referred to as atomizers. Jet nebulizers use a compressed gas (e.g., air or oxygen) to aerosolize a liquid medicine when released there through at high velocity. The resulting aerosolized droplets of therapeutic solution or suspension are then inhaled by a user for treatment. The compressed gas may be pre-compressed in a storage container or may be compressed on-demand by a compressor in the nebulizer.

Ultrasonic wave nebulizers rely on an electronic oscillator to generate a high frequency ultrasonic wave that, when directed through a reservoir of a therapeutic suspension of solution, aerosolized the medicine for inhalation.

Vibrating mesh nebulizers use the vibration of a membrane having thousands of holes at the top of the liquid reservoir to aerosolize a fine-droplet mist for inhalation. Vibrating mesh nebulizers avoid some of the drawbacks of ultrasonic wave nebulizers, offering more efficient aerosolization with reduced treatment times and less heating of the liquid being nebulized.

Treatment of a viral infection is achieved using a synergistic composition of acetic acid and hypochlorous acid. The acetic acid component is particularly effective for penetrating into tissues, while the hypochlorous acid is particularly effective for treating infection on the outer surface of tissue. As described above, these compositions are effective for treating the respiratory tract and for preventing respiratory infection.

The disclosed compositions are particularly effective because balancing the concentrations of hypochlorous acid and acetic acid with NaCl allows safe treatment of viruses. The precise balance depends on the formulation, the treatment site, and even the desired amount of surface penetration. The hypochlorous acid can be present in about 5 ppm up to about 1000 ppm or more. Different uses, different delivery methods, and types of tissue may require higher or lower concentrations. The acetic acid may be present at about 0.1% up to about 5.0% or more, and preferably about 1.0%. By balancing the two components, the composition can have the dual effect of treating at the surface and beneath the surface of the tissue to which it is applied.

In the case that the OC is hypochlorous acid HOCl, an instant composition having a concentration of about 15-60 ppm of the OC is normally sufficient for treatment of infected lungs. In the case that the OC is chlorine dioxide OCl₂, a concentration of 0.1-5 ppm is usually sufficient.

In some cases, to fully destroy the virus or to prevent the virus from entering the respiratory tract, the composition should be in contact with it for a prolonged period, ranging from a few seconds, to several minutes, to an hour or more. Accordingly, in certain embodiments, the composition is in the form of a gel, which allows longer contact times with the infection site.

The use of the composition in combination with a known antiviral treatment may increase the efficacy of the compositions. In some embodiments, methods of the invention further comprise administration (simultaneously or sequentially with compositions of the invention) of one or more doses of an antiviral substance. These may include, but are not limited to, acyclovir, adefovir, adamantine, boceprevir, brivudin, cidofovir, emtricitabine, entecavir, famciclovir, fomivirsen, foscarnet, ganciclovir, lamivudine, penciclovir, telaprevir, telbivudine, tenofovir, valacyclovir, valganciclovir, vidarabine, m₂ inhibitors, neuraminidase inhibitors, interferons, ribavirin, nucleoside reverse transcriptase inhibitors, non-nucleoside reverse transcriptase inhibitors, non-structural protein 5a (ns5a) inhibitors, chemokine receptor antagonist, integrase strand transfer inhibitors, protease inhibitors, and purine nucleosides.

Compositions of the invention are also useful in combination with a known antimicrobial treatment. In some embodiments, methods of the invention further comprise administration (simultaneously or sequentially with compositions of the invention) of one or more doses of an antibiotic substance, including, but not limited to, ciprofloxacin, beta-lactam antibiotics like ampicillin or carbapenems, azithromycin, cephalosporin, doxycycline, fusidic acid, gentamycin, linezolid, levofloxacin, norfloxacin, ofloxacin, rifampin, tetracycline, tobramycin, vancomycin, amikacin, deftazidime, cefepime, trimethoprim/sulfamethoxazole, piperacillin/tazobactam, aztreanam, meropenem, colistin, or chloramphenicol.

In some embodiments, methods of the invention further comprise administration of one or more doses of an antibiotic substance from an antibiotic class including, but not limited to, aminoglycosides, carbacephem, carbapenems, first generation cephalosporins, second generatin cephalosporins, third generation cephalosporins, fourth generation cephalosporins, glycopeptides, macrolides, monobactam, penicillins, polypeptides, quinolones, sulfonamides, tetracyclines, lincosamides, and oxazolidinones. In some embodiments, methods of the invention comprise administration of a nonantibiotic antimicrobial substance, including but not limited to sertraline, racemic and stereoisomeric forms of thioridazine, benzoyl peroxide, taurolidine, and hexitidine.

The dosing regimen of the composition may include the amount, frequency, and duration of exposure to the composition. The dosing regimen may depend on the severity of the infection, or on a regimen prescribed for treatment or for prevention of the viral infection.

The composition may be administered in a single daily dose or in multiple doses, e.g., 2, 3, 4, or more doses, per day. The subject receiving the composition may be exposed to the composition for periods of hours or of minutes. The duration of exposure may depend on the frequency, amount, or even of the severity of the infection.

The total daily amount of API formed in the instant solution from the solid precursors may be in the range 0.01-1000 mg, depending of the nature of the OC. The actual dosage may vary depending upon the specific composition administered, the mode of administration, and other factors known in the art.

The composition may be administered to any member of the respiratory tract, such as the respiratory epithelium, nasal cavity, nasal epithelium, pharynx, esophagus, larynx, epiglottis, trachea, carina, bronchi, bronchioles, or the lungs. Administering the composition to the respiratory tract treats prevents any disease or disorder that is transmitted by a virus.

In certain other embodiments, the compositions of the invention can be used to disinfect whole rooms, facilities medical devices and surgical instruments, for example. Supplies of medical devices are often initially sterile, but may require additional or subsequent cleaning and disinfection or sterilization. In particular, sterilization or disinfection of reusable medical devices prior to reuse employing any known technique is especially important. Compositions can be applied to the medical device using. For example, the composition can be applied by wiping or spreading it onto the surface of the device, by spraying an aerosol or mist form of the composition onto the device, by dipping the device into a vessel containing a volume of the composition, or by placing the device into a flow of the composition such as from a faucet. Additionally or alternatively, medical devices and surgical instruments may also be stored submerged in the composition and removed at the time of use.

Some of the disclosed compositions contain acetic acid at 2% or greater, and when in combination with the OC have proven to be safe and effective for treating skin and other tissues. The OC in these compositions has been found to have a modulating effect of the acetic acid. This allows the compositions to take advantage of the disinfecting properties of acetic acid without causing harm to the tissue.

### Use of the Invention for COVID-19 Treatment, and Other Respiratory Infectious Diseases

As previously described, in one aspect, the present invention is directed to a disinfectant composition developed to provide a safe and effective means of treating and preventing the spread of respiratory infections, including SARS-COV-2.

Compositions for use in treating SARS infections comprise a hypochlorous acid-based, nebulized broad-spectrum antiviral and antibacterial inhalation solution. More specifically, the formulation includes hypochlorous acid (HOCl) (25 ppm to 200 ppm) that has been stabilized with acetic acid (approximately 0.25%), resulting in sustainable concentrations of HOCl with positive antimicrobial effects. The addition of acetic acid increases HOCl stability, thus making it possible to develop a treatment with extended shelf-life. Furthermore, the composition is formulated with increased pH of 5.5 and isotonicity to thereby increase tolerability within airways.

Compositions of the present invention have unique virucidal properties, especially on enveloped viruses, and provides superior antiviral activity. Accordingly, such a composition may be particularly useful for the treatment, and prevention of, for example, COVID-19. More specifically, SARS-COV-2 and many other viruses have surface proteins (i.e., spike proteins), which are referred to as “door openers” into human cells in the respiratory system. These spike proteins comprise —SH groups vulnerable to oxidation by HOCl. Low concentrations of HOCl likely oxidizes extracellular —SH groups (e.g., viral spike proteins), while being harmless to normal tissue and intracellular enzymes. As such, the antiviral effect of the composition of the present invention can destroy viral particles in the respiratory tract upon first exposure, during infection, and when virions are intracellular and subsequently released by the human airway cells. Therefore, the unique virucidal properties of the composition of the present invention, especially on enveloped viruses, makes it a powerful potential tool in the ongoing efforts to prevent the spread of the coronavirus. Such a composition can reduce duration of the disease and severity of symptoms amongst a broad population of COVID-19 patients, particularly at a time of unprecedented need, given the virulence of coronavirus throughout the world.

Table 1 (below) provides a listing of the components of the composition of the present invention, which consists of 25 ppm-200 ppm HOCl+0.25% acetic acid.

TABLE 1
Formulation of isotonic 200 ppm hypochlorous
acid and 0.25 acetic acid, isotonic, pH 5.5

| | | Amount in | | |
|---|---|---|---|---|
| | | Final Solution | | |
| Raw material | CAS No. | (weight %) | Function | Supplier |
| Sodium | 7681-52-9 | 0.01 | Active Substance | Aug. Hedinger* |
| Hypochlorite | | | | GmbH & Co |
| Acetic Acid, | 64-19-7 | 0.25 | pH regulator | Sigma |
| glacial ≥99.5% | | | | Aldrich/Merck |
| Sodium | 1310-73-2 | Added to pH | pH adjuster | Sigma |
| Hydroxide | | 5.5 ± 0.2 | | Aldrich/Merck |
| Sodium | 7647-14-5 | 0.75 | Osmolarity adjuster | Sigma |
| Chloride | | | | Aldrich/Merck |
| Purified Water | 7732-18-5 | Added to 100% | Solvent | Fargon Nordic |

Inert gas: Argon (100%)
*The Sodium Hypochlorite solution may be changed to another GMP producer.

The active ingredient in preferred compositions of the invention is hypochlorous acid (HOCl). This active ingredient is derived from sodium hypochlorite, which is produced as an aqueous solution from the reaction of gaseous Cl2 with water at alkaline pH. A 3% NaOCl is produced and added to the final IS to reach a maximum of 200 ppm (0.01% w/w) HOCl. The other ingredients of the composition include the following: Sodium Hydroxide, Ph.Eur./USP-NF grade, 0.1M solution added to required pH (5.5); pH stabilizer Acetic Acid, Ph.Eur./USP-NF grade glacier, 0.25%; Osmolarity adjuster Sodium Chloride, Ph. Eur./USP-NF grade, added to reach isotonic formulation (303mOsm); and Purified Water, water purified through Reverse Osmosis and deionized by Ion Exchange process or according to Ph.Eur./USP-NF monograph.

A preferred clinical dosage for the composition is 5 mL of 25-100 ppm hypochlorous acid. The final product also contains 0.25% acetic acid buffer. As such, the solution contains more than 99.1% HOCl and less than 0.9% OCl—. HOCl is the active substance in IS and has been found to be 80 times more effective as a sanitizing agent compared to an equivalent concentration of OCl—. Therefore, HOCl serves the dual effect in IS of being the API and acting as an antimicrobial agent to inhibit the growth of microorganisms in the final product. The composition may be presented in plastic PET vials/bottles. Before administration to the patient, the composition is transferred to a nebulizer/inhalation device reservoir. This transfer is done in the clinic. After transfer to the nebulizer, the solution is administered immediately (within 1-2 h) to the patient through liquid aerosol delivery. The patient should receive 5 mL of nebulized composition.

Compositions for viral administration are typically single-dose administration and are delivered to the respiratory tract by nebulization, using, for example, PARI BOY. The nebulizer PARI BOY Classic Inhalation System, containing PARI BOY Classic Compressor, PARI LC SPRINT nebulizer. To obtain relevant deposition of the test solution in the lower and upper airways, the nebulizer will be equipped with a PARI SMARTMASK. It should be noted that other nebulizers and inhalers may be used.

HOCl is produced by the body's own immune cells, i.e., neutrophils and monocytes/macrophages. It is a powerful oxidizing agent that chlorinates and oxidizes molecular structures, especially those with thiol, thiol-ether, and amino groups (e.g., proteins, fatty acids), leading to denaturation and loss of normal function of a wide array of microbes. HOCl is considered by the FDA to be “the form of free available chlorine that has the highest bactericidal activity against a broad range of microorganisms.” HOCl is a strong oxidizing agent, however, in low concentrations (<0.1%), it is very well tolerated and safe in wound care applications.

### INCORPORATION BY REFERENCE

Any and all references and citations to other documents, such as patents, patent applications, patent publications, journals, books, papers, web contents, that have been made throughout this disclosure are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

### EXAMPLES

### Example 1: General Procedure for Preparation of Dry, Air Free Solid Mixtures of API-P and NaCl for Loading into a Multi-Compartment Device

### Calcium

1a. Production of Dry Powder Comprising 50 ppm Sodium Hypochlorite in Sodium Chloride

In 8.95 g of dry NaCl (mw: 58.44 g/mol), 50 mg of dry sodium hypochlorite (mw: 74.44 g/mol) was blended to a homogenous mixed powder and stored under air free and dry conditions in containers shielded from light. An aliquot of 90 mg of the powder is loaded into compartment 1 of the multi-compartment device.

1b. Production of Dry Powder Comprising 100 ppm Sodium Hypochlorite in Sodium Chloride

In 8.90 g of dry NaCl (mw: 58.44 g/mol), 100 mg of dry sodium hypochlorite (mw: 74.44 g/mol) was blended to a homogenous mixed powder and stored under air free and dry conditions in containers shielded from light. An aliquot of 90 mg of the powder is loaded into compartment 1 of the multi-compartment device.

1c. Production of Dry Powder Comprising 200 ppm Sodium Hypochlorite in Sodium Chloride

In 8.8 g of dry NaCl (mw: 58.44 g/mol), 200 mg of dry sodium hypochlorite (mw: 74.44 g/mol) was blended to a homogenous mixed powder and stored under air free and dry conditions in containers shielded from light. An aliquot of 90 mg of the powder is loaded into compartment 1 of the multi-compartment device.

1d. Production of Dry Powder Comprising 500 ppm Sodium Hypochlorite in Sodium Chloride

In 8.5 g of dry NaCl (mw: 58.44 g/mol), 500 mg of dry sodium hypochlorite (mw: 74.44 g/mol) was blended to a homogenous mixed powder and stored under air free and dry conditions in containers shielded from light. An aliquot of 90 mg of the powder is loaded into compartment 1 of the multi-compartment device.

1e. Production of Dry Powder Comprising 25 ppm Calcium Dihypochlorite in Sodium Chloride

In 8.975 g of dry NaCl (mw: 58.44 g/mol), 25 mg of dry calcium hypochlorite (mw: 142.98 g/mol) was blended to a homogenous mixed powder and stored under air free and dry conditions in containers shielded from light. An aliquot of 90 mg of the powder is loaded into compartment 1 of the multi-compartment device.

1f. Production of Dry Powder Comprising 50 ppm Calcium Dihypochlorite in Sodium Chloride

In 8.975 g of dry NaCl (mw: 58.44 g/mol), 50 mg of dry calcium hypochlorite (mw: 142.98 g/mol) was blended to a homogenous mixed powder and stored under air free and dry conditions in containers shielded from light. An aliquot of 90 mg of the powder is loaded into compartment 1 of the multi-compartment device.

1g. Production of Dry Powder Comprising 100 ppm Calcium Dihypochlorite in Sodium Chloride

In 8.9 g of dry NaCl (mw: 58.44 g/mol), 100 mg of dry calcium hypochlorite (mw: 142.98 g/mol) was blended to a homogenous mixed powder and stored under air free and dry conditions in containers shielded from light. An aliquot of 90 mg of the powder is loaded into compartment 1 of the multi-compartment device.

1h. Production of Dry Powder Comprising 100 ppm Calcium Dihypochlorite in Sodium Chloride

In 8.9 g of dry NaCl (mw: 58.44 g/mol), 100 mg of dry calcium hypochlorite (mw: 142.98 g/mol) was blended to a homogenous mixed powder and stored under air free and dry conditions in containers shielded from light. An aliquot of 90 mg of the powder is loaded into compartment 1 of the multi-compartment device.

1i. Production of Dry Powder Comprising 100 ppm Calcium Dihypochlorite in Sodium Chloride

In 8.9 g of dry NaCl (mw: 58.44 g/mol), 100 mg of dry calcium hypochlorite (mw: 142.98 g/mol) was blended to a homogenous mixed powder and stored under air free and dry conditions in containers shielded from light. An aliquot of 90 mg of the powder is loaded into compartment 1 of the multi-compartment device.

1j. Production of Dry Powder Comprising 1 ppm Sodium Chlorite in Sodium Chloride

In 89.99 g of dry NaCl (mw: 58.44 g/mol), 10 mg of dry sodium chlorite (mw: 90.44 g/mol) was blended to a homogenous mixed powder and stored under air free and dry conditions in containers shielded from light. An aliquot of 90 mg of the powder is loaded into compartment 1 of the multi-compartment device.

1k. Production of Dry Powder Comprising 5 ppm Sodium Chlorite in Sodium Chloride

In 89.99 g of dry NaCl (mw: 58.44 g/mol), 50 mg of dry sodium chlorite (mw: 90.44 g/mol) was blended to a homogenous mixed powder and stored under air free and dry conditions in containers shielded from light. An aliquot of 90 mg of the powder is loaded into compartment 1 of the multi-compartment device.

1l. Production of Dry Powder Comprising 10 ppm Calcium Chlorite in Sodium Chloride

In 89.99 g of dry NaCl (mw: 58.44 g/mol), 100 mg of dry calcium chlorite (mw: 157.89 g/mol) was blended to a homogenous mixed powder and stored under air free and dry conditions in containers shielded from light. An aliquot of 90 mg of the powder is loaded into compartment 1 of the multi-compartment device.

### Example 2: General Procedure for Preparation of 1 L Stock Solutions of Activator for Low-Volume Aliquot Loading into a Multiple Compartment Device

2a. Acetic Acid Activator Stock Solution (0.125%, pH 2.95)

In 998.75 mL of sterile water, 1.25 mL of acetic acid (mw: 60.05 g/mol) was dissolved.

2b. Acetic Acid Activator Stock Solution (0.125%, pH 4.3)

In 998.75 mL of sterile water, 1.25 mL of acetic acid (mw: 60.05 g/mol) was dissolved. The pH was adjusted to 4.3 using 10 N NaOH.

2c. Acetic Acid Activator Stock Solution (0.25%, pH 4.3)

In 998.75 mL of sterile water, 2.5 mL of acetic acid (mw: 60.05 g/mol) was dissolved. The pH was adjusted to 4.3 using 10 N NaOH.

2d. Acetic Acid Activator Stock Solution (0.25%, pH 5.0)

In 998.75 mL of sterile water, 2.5 mL of acetic acid (mw: 60.05 g/mol) was dissolved. The pH was adjusted to 5.0 using 10 N NaOH.

2e. Acetic Acid Activator Stock Solution (1%, pH 4.3)

In 998.75 mL of sterile water, 10 mL of acetic acid (mw: 60.05 g/mol) was dissolved. The pH was adjusted to 4.3 using 10 N NaOH.

2f. Acetic Acid Activator Stock Solution (2%, pH 4.3)

In 998.75 mL of sterile water, 20 mL of acetic acid (mw: 60.05 g/mol) was dissolved. The pH was adjusted to 4.3 using 10 N NaOH.

2g. Acetic Acid/Sodium Acetate Activator Stock Solution (0.1 M, pH 5.0)

In 800 mL of distilled water, 5.772 g of sodium acetate (mW: 82 g/mol), 1.778 g of acetic acid (mw: 60.05 g/mol) was added to the solution. The pH was adjusted to 5.0 using 10N HCl or 10 N NaOH, and distilled water was added until the volume was 1 L.

2h. Isotonic Acetic Acid Activator Stock Solution (0,125%, pH 2.95)

In 998.75 mL of sterile water, 1.25 mL of acetic acid (mw: 60.05 g/mol) and 8.4 g NaCl (mw: 58.44 g/mol) was added.

2i. Isotonic Acetic Acid Activator Stock Solution (0.125%, pH 4.3)

In 998.75 mL of sterile water, 1.25 mL of acetic acid (mw: 60.05 g/mol) and 8.4 g NaCl (mw: 58.44 g/mol) was added. The pH was adjusted to 4.3 using 10 N NaOH.

2j. Isotonic Acetic Acid Activator Stock Solution (0.25%, pH 4.3)

In 998.75 mL of sterile water, 2.5 mL of acetic acid (mw: 60.05 g/mol) and 8.4 g NaCl (mw: 58.44 g/mol) was added. The pH was adjusted to 4.3 using 10 N NaOH.

2k. Isotonic Acetic Acid Activator Stock Solution (0.125%, pH 5.0)

In 998.75 mL of sterile water, 1.25 mL of acetic acid (mw: 60.05 g/mol) and 8.4 g NaCl (mw: 58.44 g/mol) was added. The pH was adjusted to 5.0 using 10 N NaOH.

2l. Isotonic Acetic Acid Activator Stock Solution (0.25%, pH 5.0)

In 998.75 mL of sterile water, 2.5 mL of acetic acid (mw: 60.05 g/mol) and 8.4 g NaCl (mw: 58.44 g/mol) was added. The pH was adjusted to 5.0 using 10 N NaOH

2m. Isotonic Acetic Acid/Sodium Acetate Activator Stock Solution (0.1 M, pH 5.0)

In 800 mL of distilled water, 5.772 g of sodium acetate (mW: 82 g/mol), 1.778 g of acetic acid (mw: 60.05 g/mol) and 8.4 g NaCl (mw: 58.44 g/mol) was added to the solution. The pH was adjusted to 5.0 using 10N HCl or 10 N NaOH, and distilled water was added until the volume was 1 L.

2n. Acetate Buffer (0.1 M, pH 5.0)

In 800 mL of sterile water, 5.772 g of sodium acetate (mW: 82 g/mol) and 1.778 g of acetic acid (mw: 60.05 g/mol) was added to the solution. The pH was adjusted to 5.0 using 10N HCl, and distilled water was added until the volume was 1 L.

2o. ACES Buffer (0.1 M, pH 6.7)

In 800 mL of sterile water, 18.22 g of N-(2-acetamido)-2-aminoethanesulfonic acid (mW: 182.2 g/mol) was added to the solution. The pH was adjusted to 6.7 using pH using 10N NaOH, and distilled water was added until the volume was 1 L.

2p. Citric Acid Solution (0.1 M, pH 2.2)

An amount of 19.2 g of citric acid (mw: 192.1 g/mol) was dissolved in 1 L of sterile water.

2q. Citrate Buffer (0.1 M, pH 6.0)

In 800 mL of sterile water, 12.044 g of sodium citrate (mW: 294.1 g/mol) and 11.341 g of citric acid (mw: 192.1 g/mol) was added to the solution. The pH was adjusted to 6.0 using 0.1 N NaOH, and distilled water was added until the volume was 1 L.

2r. ADA Buffer (0.1 M, pH 6.6)

In 800 mL of sterile water, 95.11 g of 2-[(2-amino-2-oxoethyl)-(carboxymethyl)amino]acetic acid (ADA, mW: 190.22 g/mol) was added to the solution. ADA dissolved when the pH was adjusted to 6.6 using pH using 10N NaOH, and distilled water was added until the volume was 1 L.

2s. EBBS Buffer Including the Dye Phenol Red (pH 7.0)

In 800 mL of sterile water, 200 mg of CaCl₂) (mW: 110.98 g/mol), 200 mg of MgSO₄-7H₂O (mW: 246.47 g/mol), 400 mg of KCl (mW: 75 g/mol), 2.2 g of NaHCO₃ (mW: 84.01 g/mol), 6.8 g of NaCl (mw: 58.44 g/mol), 140 mg NaH₂PO₄H₂O (mw: 138 g/mol), 1 g D-Glucose (Dextrose) (mw: 180.16 g/mol) and 10 mg phenol red Phenol Red (mw: 354.38 g/mol) was added to the solution. The pH of the solution was adjusted to 7.0 or another desired pH using HCl or NaOH.

2t. Sterile Isotonic Oxygenated Water

A stock volume of 1 L of sterile water saturated with oxygen is added 9 g of NaCl and stored at room temperature in a sealed bottle shielded from light.

### Example 3: Instant Preparation of Ready to Use Disinfectant Formulations from Solid Salts of Oxidized Chlorine Combined with Solutions from Example 1

### Example 3.1: Non-Limiting Steps of a General Procedure

1. An aliquot of 90 mg of any of the powders from example 1 is loaded into compartment 4 of the multi-compartment device.
2. An aliquot of 10 mL of any of the activator solutions from Example 2 is loaded into compartment 4 of the multi-compartment device.
3. To generate the main product according to the invention, the seal, barrier or port 3 according to FIG. 1 between the screw cap and compartment 4 is broken or opened to mix the contents in compartment 1 with the solution in compartment 4, followed by gently squeezing or shaking to generate the disinfectant solution. The resulting solution can be taken out through the opening after removing the screw cap on the multi-compartment device, and are now ready to use. The isotonic solutions have a pH in the interval 4 to 9, preferably between 5 and 6, is generally used for antimicrobial purposes.
4. Optionally, depending on the intended use, a water-soluble dye in solid form with a color that varies with the oxidation state of the API (ROD), in a precalculated amount to generate a concentration of the dye in the concentration range 0.01-1000 ppm, is optionally loaded into compartment 9 of the multi-compartment device, and the procedure in 3 is repeated including mixture of compartments 1, 4 and 9.
5. Optionally, depending on the intended use, a precalculated amount an amino acid as a stabilizer of the API, preferably taurine in the same concentration as the API, is optionally loaded into compartment 5 of the multi-compartment device, and the procedure in 3. is repeated including mixture of compartments 1, 4 and 5.
6. Optionally, depending on the intended use, e.g. for skin or wound applications, an amount of a water-soluble viscosity enhancer (VE) that cannot be oxidized by the API, precalculated to gain a concentration of VE in the final solution in the concentration range 0.01-25%, is loaded into compartment 5 of the multi-compartment device. A VE concentration of 0.01-0.1% generates a viscous but fluid solution, while 0.3-1% produce a gel. The dispersion of the VE in the solution from step 3, 4 and/or 5 is converted to a viscous solution or a gel using a Silverson Mixer or an Ystral Mixer, and used on site for skin or wound applications. The viscous solution or a gel have increased stability because of slower motions of molecules and may be packed into soft bags, bottles protecting the solution or gel from air and light for later use.

### Example 3.2: General Procedure for Preparation of Reconstitutable Hypochlorous Acid-Based Composition

1. An aliquot of reconstitutable agent(s) is mixed with a diluent to thereby form a ready to use disinfectant formulation in accordance with the present invention, including, but not limited to, the hypochlorous acid-based broad-spectrum antimicrobial solution described herein. The reconstitutable agent(s) and diluent are mixed in a multi-compartment device, similar to the device illustrated in FIG. 1 and described herein.
2. The reconstitutable agents may include, but are not limited to, dry powdered agents, including any of the powders from Example 1, as well as any of the activator solutions from Example 2 that have been prepared into dry powder form. For example, in one embodiment, one of the reconstitutable agents may include calcium hypochlorite in dry powder form (provided in a first compartment of a multi-compartment device) and a second reconstitutable agent may include sodium acetate in dry powder form (provided in a second compartment of the multi-compartment device). A diluent, such as water (or other aqueous medium), is provided in a third compartment.
3. Each of the reconstitutable agents (the powdered calcium hypochlorite and powdered sodium acetate) and the diluent are maintained in separate respective compartments of the mixing device until a user is ready to combine and mix the agents and diluent to form an antimicrobial solution as described herein. For example, the device may include one or more breakable seals separating one or more of the compartments from one another, such that a user need only break the seal to thereby initiate mixing of the reconstitutable agents and diluent together. Once the agents and diluent are mixed, a user need only shake the device to agitate and adequately combine the agents and diluent. The device may include one or more filters for filtering the diluent prior to mixing with the reconstitutable agents and/or filtering the resulting antimicrobial solution prior to use. For example, the device may come preloaded with the reconstitutable agents but without the diluent (i.e., water). Accordingly, in the field and at the site of use, a user need only provide water (i.e., from a water source) to the device, at which point a filter may filter out any impurities in the water to provide an adequate diluent. Furthermore, upon producing the resulting antimicrobial solution, the solution may pass through a one way filter when a user applies the solution to the site of intended use (i.e., wound irrigation, hand disinfectant, inhalation solution, countermeasures towards biological and chemical weapons, and the like. Accordingly, the filter prevents contaminated solution from reentering the device during use.
4. Optionally, depending on the intended use, a colorimetric indicator may be added to the composition to provide a visual indication of the level of antimicrobial effects in the solution. The colorimetric indicator may include a dye, for example, such as a reduction-oxidation dye (ROD), such that the color and intensity of color of the dye is dependent on an oxidation state of the oxidized chlorine compound. For example, if the standard half-cell potential of the ROD has a lower positive value than oxidized chlorine (OC), the color of the formulation will be maintained as long as the OC is active. Thereby, the color provides a visual clue in the region wherein the formulation has been applied and where there is active OC. Further, employment of the opposite type of indicator, where the color appears when the oxidizing power of the OC is vanishing, is also useful. The mixing device itself may also include a color scale of sorts that provides various shades of color indicating levels of concentration and appropriate uses/applications for such levels of concentration.
5. Various techniques to prepare dry powders are known and practiced. Such techniques include lyophilization, spray-drying, spray-freeze drying, bulk crystallization, vacuum drying, and foam drying. Lyophilization (freeze-drying) is often a preferred method used to prepare dry powders (lyophilizates) containing proteins. Various methods of lyophilization are well known to those skilled in the art. The lyophilization apparatus and process applies a vacuum that converts liquid portions of a composition into a solid which is subject to a sub-atmospheric pressure to create a vapor. The vapor is drawn from the lyophilization chamber through vapor passages and exhausted to regions external of the lyophilizing apparatus. The lyophilizing process reduces the liquid composition to a dried powdery or granular form. In particular, freeze drying, or lyophilization, is a dehydration technique. It takes place while a product is in a frozen state (ice sublimation under a vacuum) and under a vacuum (drying by gentle heating). These conditions stabilize the product, and minimize oxidation and other degradative processes. The conditions of freeze drying permit running the process at low temperatures, therefore, thermally labile products can be preserved. Freeze drying has become an accepted method of processing heat sensitive products that require long term storage at temperatures above freezing
6. Steps in freeze drying include pretreatment, freezing, primary drying and secondary drying. Pretreatment includes any method of treating the product prior to freezing. This may include concentrating the product, formulation revision (i.e., addition of components to increase stability and/or improve processing), decreasing a high vapor pressure solvent or increasing the surface area. Methods of pretreatment include: freeze concentration, solution phase concentration, and formulating specifically to preserve product appearance or to provide lyoprotection for reactive products.
7. Accordingly, by providing the reconstitutable agents and diluent(s) within a mixing device, as described herein, the resulting antimicrobial solution can be produced at the desired time and at the desired site of use without facing storage degradation. As previously described herein, certain antimicrobial solutions may typically have a relatively short shelf-life, as they may contain compounds that degrade rapidly and lose their effectiveness. As such, some formulations may require refrigeration and special packaging, or require immediate use upon being produced. Such special treatment, however, adds to operating costs and complicates storage, particularly in areas where such storage is not available. Accordingly, the present invention allows for antimicrobial solutions to be produced by a user at a point of use (e.g., in the field) where access to such solutions is critical, such as in military situations or the like.

### Example 4: In Vitro Anti-Biofilm Effect of Example 3 Test Solutions of HOCl and Acetic Acid

Three different test solutions were generated form the multi-compartment device. All three test solutions are generated as described in example 3.1 from the multicompartment device, loaded with 90 mg of dry powder comprising 200 ppm sodium hypochlorite in sodium chloride (example 1c) in compartment 1. Three aliquots of 10 mL of acetic acid solutions from (0.125%, pH 4.3, example 2b) in compartment 4 in three different multicompartment-devices. Solution 1: (0.25%, pH 4.3, example 2c), Solution 2: (1%, pH 4.3, example 2e), Solution 3: (2%, pH 4.3, example 2f).

### Experimental Setup

Test organisms: Pseudomonas aeruginosa or Staphylococcus aureus wild-type strains Biofilm type: 48 hours- or 24 hours-old biofilms grown on semipermeable membranes placed on solidified medium supplemented with 0.5% glucose. In the case of 48 h-old biofilms, the membranes with biofilms were transferred onto fresh plates after 24 h.

Initial viable cell amount: 5×10⁹ colony forming units (CFUs)

Treatment method: Membranes with biofilms were transferred to new plates. Eight-10 layers of sterile gauze were placed on the second membrane, and 1 ml of antimicrobial solution was pipetted on the gauze layers. The treatment was carried out at room temperature for 2-to-3h, or 4-to-6h. In the case of the 4-to-6 h treatments, the gauze layers were replaced with fresh gauze layers with 1 ml sample solution 2 or 3 h after the treatments had been initiated.

Evaluation method: The gauze layers were discarded, and each membrane with biofilms was transferred into a 15 ml tube containing 5 ml 0.9% NaCl, vortexed for 10 sec., sonicated in an ultrasound bath for 10 min, and vortexed again for 10 sec. Ten-fold serial dilutions were made, and 10 ul of each dilution was spot-plated on LB plates for viable CFU counting.

### Results and Conclusions

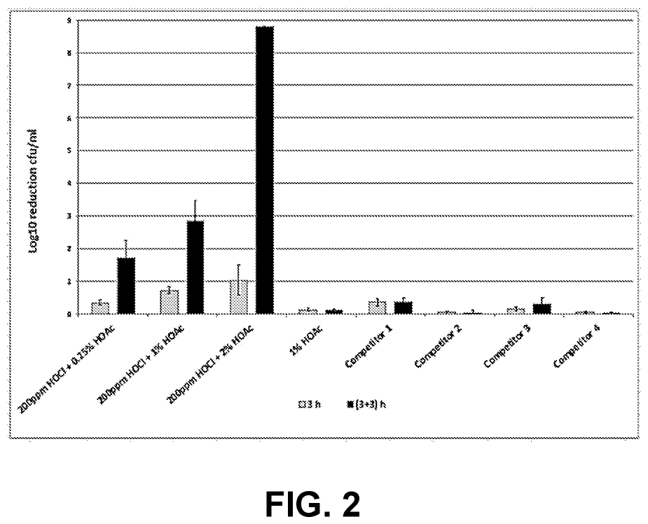

FIG. 2 shows the results obtained using the sample solutions. Increasing the HAc concentrations from 0.25% to 1% and 2% in a 200 ppm HOCl solution gradually increased the killing of S. aureus biofilms. The effect of 1% acetic acid alone had only minor effect on the biofilm. The three test solutions were compared to 4 different competing wound healing products on the market which all showed only minor effects on the S. aureus biofilms. An even stronger effect was shown for biofilms from P. Aeruginosa It is concluded that hypochlorous acid and acetic acid at pH 4.3 acts synergistically and efficiently at concentrations that have shown to be safe in other studies.

### Example 5: In Vivo Toxicity Studies

### Example 5.1:7 Day Inhalation Toxicity Study in Rats

A 7 day inhalation toxicity study in rats is performed as described by Kogel et al in 2913 in https://www.pmiscience.com/resources/docs/default-source/default-document-library/2013_ukogel_ict_poster.pdf?sfvrsn=d6a9f606_0. The rat inhalation study is performed according to the Organization for Economic Cooperation and Development (OECD). The test solution is generated as described in example 3.1 from the multicompartment device, loaded with 90 mg of dry powder comprising 100 ppm sodium hypochlorite in sodium chloride (example 1 b) in compartment 1 and an aliquot of 10 mL of acetic acid solution (0.125%, pH 4.3, example 2b) in compartment 4. Test Guideline 412, Sprague-Dawley rats is exposed to filtered fresh air (sham) as a reference, or the test solution. Care and use of the animals is in accordance with the American Association for Laboratory Animal Science Policy (1996). All animal experiments are approved by the Institutional Animal Care and Use Committee (IACUC). The histopathological evaluation is performed at defined anatomical sites of the nose and of the left lung according to a defined grading system. Free lung cells are determined in bronchoalveolar lavage fluid by flow cytometry, and inflammatory mediators are measured by multi-analytes profiling (MAP). For the Systems Toxicology approach, RNA samples from specific sites in the respiratory tract are obtained, i.e., respiratory nasal epithelium (RNE) and lung. For lung RNA isolation, respiratory epithelium of main bronchus and lung parenchyma is separated by Laser Capture Microdissection (LCM) and further processed, and analyzed on whole genome Affymetrix microarrays (GeneChip® Rat Genome 230 2.0 Array). No major perturbations are found related to inflammation, cell stress, cell proliferation in bronchi or lung parenchyma.

### Example 6: Treatment of Mastitis

For applications where a color indicator in step 4 can add information in the therapeutic procedure, e.g. in or for indication of the oxidative activity of the API, the compartment comprising the ROD is included in the procedure.

### Example 7: Clinical Antiviral Therapy

The medicine cup of Gima Aerosol Corsia Nebulizer is loaded with 5 mL of the test solution generated as described in example 3.1 from the multicompartment device, loaded with 90 mg of dry powder comprising 1 ppm sodium chlorite in sodium chloride (example 1j) in compartment 1 and an aliquot of 10 mL of citric acid solution (0.1 M, pH 2.2, example 2p) in compartment 4. The mouth of a patient with a coronavirus lung infection is attached to the hose and the face mask attached to the nebulizer, which is started. After 10-15 minutes of breathing, the fluid is used up, and the nebulizer is turned off. The patient is monitored for several hours to secure that no side effects of the treatment is taking place. The mucosa and cilia of the patient is investigated for potential side effects.

### Example 8: Pharmacology of Inhalation Solution (IS)

The virus-inactivating properties of the inhalation solution (IS) of the composition against modified vaccinia virus Ankara (MVA) have been investigated. The IS products at 50, 100, and 200 ppm HOCl (pH 5.5) (and diluted 50% solutions) showed virus inactivation properties suggesting that the lowest concentration of the IS product showing virus inactivation was at 25 ppm HOCl. Further dilutions were tested and diluted solutions with concentrations of 5, 10 and 20 ppm did not show any virus inactivation and effect suggesting that the non-active lower range was demonstrated. IS products with 50, 100 and 200 ppm, HOCl (pH 5.5) demonstrated antiviral activity against the enveloped DNA vaccinia virus for all tested HOCl concentrations. Products that have antiviral activity against the vaccinia virus are considered active against all enveloped viruses, including SARS-COV-2. In a separate study, IS has been shown to inactivate SARS-CoV-2 between 10 and 200 ppm HOCl.

As for antibacterial activity, overnight cultures of S. aureus and P. aeruginosa were grown for 2 and 24 hours, respectively, to test IS against both planktonic and biofilm growing bacteria. Full effect was seen for 50 ppm HOCl IS for P. aeruginosa and S. aureus (though 100 ppm HOCl IS for S. aureus biofilm).

In summary, the IS products with HOCl concentrations between 50 and 200 ppm show virus inactivation in two different MVA in vitro tests. After dilutions of the test products, the lowest concentration showing antiviral activity was at 25 ppm HOCl and the lowest diluted concentrations tested showing no antiviral activity were 5, 10 and 20 ppm HOCl. From these experiments the antiviral effective concentration range was between 25 and 200 ppm HOCl. IS has been shown to inactivate SARS-COV-2 in various concentrations.

### Example 8.1: Antiviral Efficacy

### Antiviral Effectiveness of HOCl Against Vaccinia Virus

Antiviral assays were performed to evaluate the virucidal activity of HOCl against modified vaccinia virus Ankara (MVA). The product used was IS containing 50, 100, and 200 ppm HOCl at the following concentrations:
Undiluted (80.0%)
Diluted with aqua bidest. (50.0%)
Diluted with aqua bidest. (10.0%)
Diluted with aqua bidest. (1.0%)—200 ppm HOCl only

The test methods involved exposing the test products (50, 100, & 200 ppm HOCl) at dilutions between 1-80% to BHK21-cells infected with MVA, as confirmed via infectivity assay. The product was in contact with MVA infected cells for either 1 or 2 minutes then an inactivation assay was performed to determine virucidal activity. Determination of cytotoxicity was also performed following product contact.

### Method

To prepare the test virus suspension, BHK 21-cells were cultivated with MEM and 10% or 2% fetal calf serum. Cells were infected with a multiplicity of infection of 0.1. The test product was tested undiluted. Due to the addition of interfering substance and test virus suspension an 80.0% solution resulted.

Infectivity was determined as endpoint titration according to EN 5.5 transferring 0.1 mL of each dilution into eight wells of a microtiter plate to 0.1 mL of freshly splitted cells (10-15×103 cells per well), beginning with the highest dilution. Microtiter plates were incubated at 37° C. in a 5% CO2-atmosphere. The cytopathic effect was read by using an inverted microscope. Calculation of the infective dose TCID50/mL was calculated with the method of Spearman and Kärber. The virucidal activity of the test disinfectant was evaluated by calculating the decrease in titer in comparison to the control titration without disinfectant. The difference is given as reduction factor (RF). According to the EN 14476, a disinfectant or a disinfectant solution at a particular concentration has virus-inactivating efficacy if the titer is reduced at least by 4 log 10 steps within the recommended exposure period. This corresponds to an inactivation of ≥99.99%. Determination of virucidal activity has been carried out according to EN 5.5. Inactivation tests were carried out in sealed test tubes in a water bath at 20° C.±1.0° C. Aliquots were retained after appropriate exposure times and residual infectivity was determined. Determination of cytotoxicity was performed according to EN 5.5.4.1. As reference for test validation a 0.7% formaldehyde solution according to EN 5.5.6 was included. Contact times were 5, 15, 30 and 60 minutes. In addition, cytotoxicity of formaldehyde test solution was determined according to EN 5.5.6.2 with dilutions up to 10⁻⁵.

### Results

All undiluted test products (i.e., 50, 100, 200 ppm HOCl) in an 80.0% assay were able to inactivate MVA after 1 minute of exposure time. The reduction factors were the following:
50 ppm HOCl: ≥5.25±0.33
100 ppm HOCl: ≥5.13±0.25
200 ppm HOCl: ≥5.25±0.33

These corresponded to an inactivation of ≥99.999%.

The 50.0% solutions were also able to inactivate MVA after 1 minute of exposure time. The reduction factors were the following:
50 ppm HOCl: ≥4.25±0.33
100 ppm HOCl: ≥4.13±0.25
200 ppm HOCl: ≥4.25=0.33

These corresponded to an inactivation of ≥99.99%.

The 10.0% solutions were not able to inactivate MVA within 1 minute of exposure time. The 1.0% solution (200 ppm HOCl) was also not able to inactivate MVA within 1 minute. In conclusion, the products for inhalation IS at 50, 100, and 200 ppm HOCl tested undiluted demonstrated activity against MVA after an exposure time of 1 minute (0.3 g/L BSA).

### Example 8.2: Antibacterial and Anti-Biofilm Efficacy

### Example 8.2.1. Antibacterial and Anti-Biofilm Efficacy of IS

An antibacterial assay was performed to evaluate the bactericidal activity of IS against P. aeruginosa and S. aureus grown for either 2 or 24 hours to represent planktonic and biofilm bacteria, respectively. The product used was the IS (i.e., with 0.25% acetic acid, pH 5.5, isotonic) at the following concentrations:
10 ppm HOCl
50 ppm HOCl
100 ppm HOCl
200 ppm HOCl
500 ppm HOCl

The product was in contact with either P. aeruginosa or S. aureus for 1 hour then an aliquot was plated and left to incubate overnight. The next day the plates were evaluated for growth and log reductions were quantified in the case of partial growth.

### Method

MH340 (P. Aeruginosa PAO1) was grown in 5 mL LB and NCTC-8325-4 (S. aureus) in 5 mL TSB in culture tubes overnight (17 hours) at 37° C., shaking at 180 rpm.

Overnight cultures were thereafter diluted 50 times and 200 μL of the diluted bacterial suspension were deposited per well in 96 rounded well microtiter plates (8 technical replicates). One microtiter plate per condition and treatment per bacteria. Two hours growth (“planktonic” bacteria)+1 hour treatment and 24 hours growth (“biofilm” bacteria)+1 hour treatment. The bacteria were incubated at 37° C. for 2 and 24 hours, respectively.

Thereafter bacteria were treated with 0.9% NaCl (control), 10 ppm HOCl, 50 ppm HOCl, 100 ppm HOCl, 200 ppm HOCl, and 500 ppm HOCl IS at 37° C. for 1 hour.

After the treatment period (one hour) 20 μL per well was spotted on LB plates and cultured at 37° C. overnight. The day after the plates were checked for growth (saline is control) or no growth.

### Results

As seen in Table 2 below, P. aeruginosa planktonic bacteria and biofilms were eradicated at lower product concentrations than S. aureus. There is full antibacterial effect of the final IS product (100 ppm HOCl) across the board in representative planktonic and biofilm S. aureus and P. aeruginosa.

TABLE 2
S. aureus and P. aeruginosa grown planktonic (2 hours) and in biofilms (24 hours)
and their response to different concentrations of HOCl in the inhaled product.

| HOCl concentration | | | | | |
|---|---|---|---|---|---|
| 0.9% | 10 | 50 | 100 | 200 | 500 |
| NaCl | ppm | ppm | ppm | ppm | ppm |

S. aureus + 1 hour treatment

| | | | | | | |
|---|---|---|---|---|---|---|
| Planktonic | + | 1-fold log | − | − | − | − |
| (2 h growth) | | reduction | | | | |
| Biofilm | + | + | 1-fold log | − | − | − |
| (24 h growth) | | | reduction | | | |

P. aeruginosa + 1 hour treatment

| | | | | | | |
|---|---|---|---|---|---|---|
| Planktonic | + | − | − | − | − | − |
| (2 h growth) | | | | | | |
| Biofilm | + | 1-fold log | − | − | − | − |
| (24 h growth) | | reduction | | | | |

Plus (+) indicates growth, minus (−) indicates no growth.

In conclusion, IS at concentrations of 10 or 50 ppm HOCl kill the common planktonic bacterial pathogens P. aeruginosa and S. aureus, respectively. IS with 50 or 100 ppm HOCl kill biofilm forms of P. aeruginosa and S. aureus, respectively.

### Example 8.2.2. Antibacterial and Anti-Biofilm Efficacy of IS and Acetic Acid

Another antibacterial assay was performed to evaluate the bactericidal activity of IS and acetic acid against P. aeruginosa and S. aureus grown for 2 hours to represent planktonic bacteria. The product used was the IS (i.e., with 0.25% acetic acid, pH 5.5, isotonic) or acetic acid alone at the following concentrations:
25 ppm HOCl
50 ppm HOCl
100 ppm HOCl
0.25% acetic acid, pH 5.5, isotonic

The product was in contact with either P. aeruginosa or S. aureus for 1 hour then an aliquot was plated and left to incubate overnight. The next day the plates were evaluated for growth and log reductions were quantified in the case of partial growth.

### Method

Diluted overnight cultures (OD of 0.5, ~10⁷ for S. aureus and ~10⁸ for P. aeruginosa) of S. aureus (NCTC-8325-4) and P. aeruginosa PAO1 (MH340) were grown in 96-well microtiter plates for 2 hours, to test antibacterial properties against planktonic gram-positive and gram-negative bacteria. Wells were thereafter treated with IS at varying concentrations of HOCl (25, 50, and 100 ppm HOCl, 0.25% acetic acid, pH 5.5, isotonic), isotonic 0.25% acetic acid (pH 5.5), and 0.9% saline (control) for one hour before harvest.

After one hour, Dey-Engley neutralizing broth (Sigma Aldrich, D3435) was added to all wells to inactivate IS and the content of the wells were diluted in 10-fold series and plated on relevant agar plates (down to 10⁻⁸). The plates were grown aerobically for 18 hours at 37° C. The CFU counts were calculated from the number of colonies in the countable dilutions to calculate log reductions. Test was run with three technical replicates of each bacterium.

### Results

As seen in Table 3 below, 25, 50 and 100 ppm HOCl IS eradicated both gram-positive (S. aureus) and gram-negative (P. aeruginosa) bacteria. Isotonic 0.25% acetic acid (pH 5.5) did not eradicate the bacteria.

TABLE 3
S. aureus and P. aeruginosa grown for two hours and thereafter
treated with IS with different concentrations of HOCl, 0.25%
acetic acid (isotonic, pH 5.5), or saline as control. Plus
(+) indicates growth, minus (−) indicates no growth

| | | | | | |
|---|---|---|---|---|---|
| | | 25 ppm | 50 ppm | 100 ppm | 0.25% acetic |
| | 0.9% | HOCl | HOCl | HOCl | acid isotonic, |
| Test solution | NaCl | IS | IS | IS | pH 5.5 |

S. aureus + 1 hour treatment

| | | | | | |
|---|---|---|---|---|---|
| Planktonic (2 h | + | − | − | − | + |
| growth) | | | | | |

P. aeruginosa + 1 hour treatment

| | | | | | |
|---|---|---|---|---|---|
| Planktonic (2 h | + | − | − | − | 1-fold log |
| growth) | | | | | reduction |

In conclusion, IS efficiently eradicates planktonic gram-positive (S. aureus) and gram-negative bacteria (P. aeruginosa) at HOCl concentrations of 25 ppm and 10 ppm, respectively. Acetic acid does not eradicate gram-positive (S. aureus) bacteria and shows minimal reduction in gram-negative bacteria (P. aeruginosa).

### Example 8.3: Anti-SARS-COV-2 Efficacy

Viral inactivation and cytotoxicity assays were performed to evaluate the virucidal activity of IS against SARS-COV-2 infected Vero E6 cells. The product used was IS at the following concentrations:
10 ppm HOCl
50 ppm HOCl
100 ppm HOCl
200 ppm HOCl

The test method involved exposing the test product at concentrations between 10-200 ppm HOCl to Vero E6 cells infected with SARS-Cov-2 for 48 hours. The cells were then stained, and the number of virus antigen positive cells were enumerated. A cell proliferation assay was performed to evaluate cytotoxicity.

### Method

Vero E6 cells/well were seeded in 96-well plates, the virus (multiplicity of infection 0.002) was added and incubated for 1 h at 37° C. or media only for non-treated controls and for cytotoxicity assays. The virus was removed and IS 10 ppm HOCl, 50, 100, or 200 ppm HOCl, either undiluted or diluted by half was added for 15 min, thereafter the assay was incubated for 48 h. The incubated cells were fixed and stained with primary antibody SARS-COV-2 spike chimeric monoclonal antibody and with secondary antibody F(ab′)2-Goat anti-Human IgG Fc Cross-Adsorbed Secondary Antibody, HRP. Single infected cells were visualized with DAB substrate and counted automatically by an ImmunoSpot series 5 UV analyzer. Cytotoxicity assays were performed using the Cell Titer AQueous One Solution Cell Proliferation Assay.

### Results

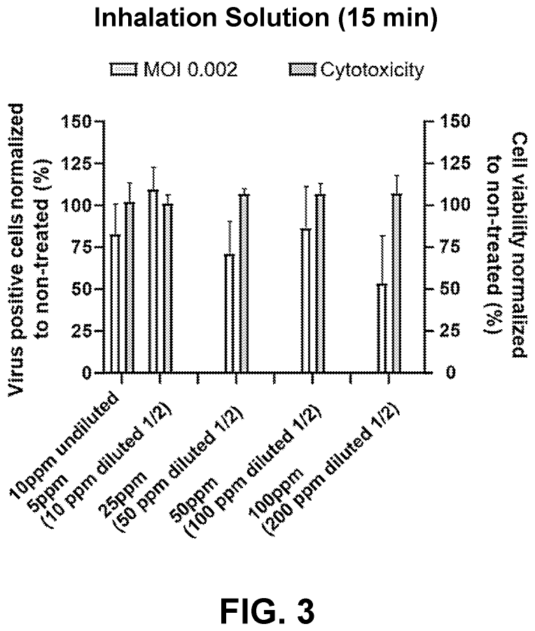

In this study, the antiviral effect of the test compound was evaluated by amount of VERO cells free of virus compared to the control. Based on the results IS lowered the amount of virus positive VERO cells, thus IS inactivated SARS-COV-2 in concentration from 10 ppm to 100 ppm HOCl without killing the VERO cells. VERO cells have been reported to be extremely fragile and not well suited to study IS thus even better antiviral activity might have been obtained with more robust cells. However, it has not been possible to run these experiments in other cell types due to the classification of SARS-COV-2 as a Class 3 microorganism. The viral inactivation and cytotoxicity results are presented in FIG. 3.

Referring to FIG. 3, each bar represents the mean with standard error of the mean (error bars). Left axis shows the number of virus antigen positive cells normalized to non-treated controls (in percentage). Right axis shows the cell viability (absorbance) normalized to non-treated controls (in percentage). MOI=Multiplicity of infection.

Note, undiluted experiments at 50, 100, and 200 ppm killed the VERO cells due to an unknown mechanism and therefore are not reported in the figure above. However, 10 ppm undiluted and 50:50 dilution of 50, 100 and 200 ppm did not kill the VERO cells and SARS-Cov-2 inactivation was observed. In conclusion, at various concentrations, IS inactivates SARS-COV-2.

### Example 9: Toxicology of Inhalation Solution (IS)

Several in vivo studies have been performed and are on-going to characterize the toxicology of IS.

Non-GLP in vivo inhalation toxicity studies in Göttingen minipigs have been performed at Ellegaard Göttingen Minipigs in Denmark. These studies include a 5-day repeated dosing study in minipigs by intubation with nebulized IS, including a recovery period of 2 or 4 weeks for selected animals. In addition, a small pilot study by intubation was performed to aid selection of dose levels for the subsequent studies. Intubation was selected as the dose route in these studies to maximize the amount of the IS that reached the lungs.

Following these studies, a further study of 5 days duration was also performed at Ellegaard Göttingen minipigs with dosing nebulized IS by mask to mimic the intended human exposure to be studied in the proposed clinical trials.

A further non-GLP Maximum Tolerated Dosage study in minipigs was performed as a preliminary study to a 14-day repeat dose GLP inhalation toxicity study in minipigs. Both studies (preliminary and main study) have dosing via mask, again to mimic as closely as possible the human administration. Due to animal welfare restrictions, the minipigs may only be dosed once per day and therefore are exposed to nebulized IS for 60 minutes to deliver the full day dosing intended for the clinical studies (i.e., 18 mL at 100 ppm) as opposed to 5 mL dosing multiple times per day.

### Example 9.1: Repeat-Dose Toxicity

The initial toxicity studies (non-GLP) were conducted at Ellegaard Göttingen Minipigs in Denmark. An additional preliminary non-GLP study was performed at Covance in England and a GLP study is on-going at Covance in England. All completed and planned repeat-dose toxicity studies are summarized in the following subsections.

### Example 9.1.1: In Vivo Inhalation Study-Intubated

Forty-two healthy young-adult Göttingen minipigs, 21 males and 21 females, 6-8 months of age, were used in this experiment. The minipigs weighed approximately 12 kg. The minipigs were bred and housed at Ellegaard Göttingen Minipigs in AAALAC International approved barrier facility housing and according to the facilities' standard for local environment, feeding, and care. The experimental protocol was approved by the Danish Animal Experiments Inspectorate (license no. 2020-15-0201-00530), and all procedures were carried out according to the Danish Animal Testing Act. The study was not performed according to GLP, however data were recorded and reported according to the documented Study Plan and to local Standard Operating Procedures.

The study was performed in two separate phases. In the first phase, 32 animals (4 males and 4 females per group) were treated for 5 days and terminated. In the second phase, a further 10 animals (5 males and 5 females) were treated at the highest dose; 1 male and 1 female were killed on Day 5 following the last treatment, and 2 males and 2 females were killed respectively after a 14 or 28-day recovery period. Both phases are summarized and reported here as a single study for convenience.

The animals were allocated to the dosing groups as follows:

### Main Phase

0.9% NaCl as control (4 males and 4 females)
50 ppm HOCl+0.25% HAc, pH 5.5, isotonic (4 males and 4 females)
100 ppm HOCl+0.25% HAc, pH 5.5, isotonic (4 males and 4 females)
200 ppm HOCl+0.25% HAc, pH 5.5, isotonic (4 males and 4 females)

### Recovery Phase

200 ppm HOCl+0.25% HAc, pH 5.5, isotonic (1 male and 1 female killed following the final dose)
200 ppm HOCl+0.25% HAc, pH 5.5, isotonic (2 males and 2 females killed after 2 weeks recovery)
200 ppm HOCl+0.25% HAc, pH 5.5, isotonic (2 males and 2 females killed after 4 weeks recovery)

Additionally, four minipigs were used in a pilot study, where three were dosed with a 500 ppm+0.25% HAc, pH 5.5, isotonic IS, whilst one received saline and acted as a control.

All minipigs were anaesthetized (with propofol potentiated by butorphanol by intravenous catheter) daily for five days to receive 5 mL nebulized product (saline for the control group) through an endotracheal tube. The minipigs were ventilated using a GE anesthesia machine at volume-controlled ventilation with a total flow of 2 L/min (50% oxygen) and a tidal volume of 10 mL/kg. Spirometry, including Pₚₑₐₖ (our major outcome parameter, to assess potential bronchoconstriction), was recorded every two minutes as well as capnometry, non-invasive blood pressure, heart rate (ECG), and temperature.

The animals were allowed at least 10 min of stabilization at the ventilator system before observations, including Pₚₑₐₖ, were recorded. The animals were monitored for 10 min as baseline measurements; thereafter the nebulization of 5 mL product was started (Aerogen Solo nebulizer, Timik Aps, Kolding, Denmark). Nebulization lasted 11-20 min (as according to manufacturer, 2-5 min/mL). After all product was nebulized, the animals were monitored for another 15 min (post-inhalation) before they could regain consciousness.

Every morning before and every afternoon after the anesthesia/inhalation, all animals were scored to assess general condition, appetite, behavior, coughing, lung function, and mobility. Blood samples were taken before the first dose and again after the last dose and evaluated for clinical pathology parameters. For recovery animals, blood was also evaluated for clinical pathology during the off-dose period.

All animals were killed on Day 5 after completion of dosing except for the recovery group animals which were killed after 2 or 4 weeks off-dose. Routine necropsy with special attention to the respiratory system was performed following euthanasia by an experienced veterinary pathologist to observe potential macroscopic signs of toxicity in situ. Lungs and mediastinal lymph nodes were weighed. Samples for histopathology were collected proximally (including the main bronchus) and distally from all seven lung lobes, from the trachea, carina, mediastinal lymph nodes, heart (right and left ventricular muscles), kidney and liver of all animals (plus 2 sentinel, untreated animals from the animal facility).

In the pilot study with 500 ppm HOCl, there was moderate ciliary loss in the respiratory epithelium, mainly in the proximal lung samples. Based on this finding, it was decided that 200 ppm HOCl would be a suitable high dose level for the main study.

In the main study, all minipigs were found to be normal at the twice daily clinical evaluations. Hematological and biochemical parameters were unremarkable for all groups at baseline (Day 1 before inhalation) and at the end of the experiment (Day 5 after inhalation). There were no findings indicative of an effect of treatment.

In the spirometry measurements, the major outcome parameter, Pₚₑₐₖ, did not differ between the different treatment groups or control in relation to and after inhalation. Further, the largest difference in Pₚₑₐₖ seen per minipig per experiment was 1 cm H₂O, which is within the limits of detection of the machine and of no clinical significance; however, for two pigs (one in the control group and one in the 200 ppm HOCl group) the differences in Pₚₑₐₖ was 2 cm H₂O. This clearly underlines that the inhalation of the nebulized products did not induce bronco-constriction. All other parameters were unaffected by treatment.

At necropsy, no apparent macroscopic signs of reaction to treatment were observed. In the first part of the study (treatment of 4+4 animals per group at 0, 50, 100, or 200 ppm, plus the pilot group of three animals dosed at 500 ppm HOCl) pathological findings related to drug exposure were local lymph node hyperplasia, loss of epithelial ciliation in the carina area (tracheal bifurcation) and main bronchi. For the main bronchi, loss of ciliation was primarily present proximally in the lung lobes, and all lobes were equally affected. Neutrophilic granulocyte infiltration was seen in mucosa and submucosa of trachea, carina and main bronchi, and the incidence followed the pattern of ciliation loss. The pathological findings related to drug exposure were present when 500 and 200 ppm HOCl were administered. However, 500 ppm resulted in the most pronounced findings. Only minimal ciliation loss was observed in single animals which received 100 ppm HOCl. All the other minor macroscopic and microscopic findings are considered either related to the daily anesthetic procedure or being incidental findings. No differences were observed between male and female animals.

In the second part of the study (animals dosed at 200 ppm and killed immediately after the last dose (1+1), or after 2 weeks recovery (2+2) or after 4 weeks recovery (2+2)), the histopathological findings in the 200 ppm HOCl group without recovery were found to be comparable to the 200 ppm group in the main study. Thus, it was confirmed that daily inhalation of 200 ppm HOCl for five days results in loss of cilia in trachea, carina area, and main bronchi. Recovery of ciliation loss was found after both 2 and 4 weeks of recovery. Hyperplasia was seen in the bronchial and bronchiolar epithelium in the recovery groups which represents signs of cellular regeneration. Neutrophilic granulocyte infiltration in mucosa and submucosa of trachea, carina, and main bronchi was not seen after recovery. The recovery groups showed an increased amount of intra-alveolar macrophages. However, a large variation was seen which, together with the low number of animals in each group, make it difficult to clearly relate the finding to the tested drug. In addition, the estimated number of intra-alveolar macrophages in the 200 ppm HOCl group in the first study was much higher than in the second. Furthermore, focal infiltration of alveolar macrophages, sometimes associated with mineralization, are reported as common findings in Göttingen minipigs. No differences were observed between female and male animals. SIS at 100 ppm HOCl (5.0 mL) was considered to be the NOAEL following dosing of minipigs by intubation.

### Example 9.1.2: In Vivo Inhalation Study-Masked

In this study, healthy minipigs were treated daily for five days with 10 mL (10 mL is added to the nebulizer, but the expected delivery was 8.8 mL, as residual volume is 1.2 mL) of the nebulized IS or nebulized saline solution (0.9% w/v NaCl, as a control) by mask covering the snout. The previously found NOAEL of 100 ppm was tested as well as 50 ppm and compared to saline control.

Twelve healthy young-adult Göttingen minipigs (6-8 months of age) were used in this experiment (31355). The minipigs (6 males and 6 females) weighed approximately 12 kg. The minipigs were bred and housed at Ellegaard Göttingen Minipigs in AAALAC International approved barrier facility housing and according to the facilities' standard for local environment, feeding, and care. The experimental protocol was approved by the Danish Animal Experiments Inspectorate (license no. 2020-15-0201-00530), and all procedures were carried out according to the Danish Animal Testing Act.

The animals were randomly divided into the following dosing groups with 4 animals (2 males and 2 females):
0.9% NaCl as control (n=4)
50 ppm HOCl+0.25% acetic acid, pH 5.5, isotonic (n=4)
100 ppm HOCl+0.25% acetic acid, pH 5.5, isotonic (n=4)

The minipigs were trained to accept the sling confinement on two occasions during the week before the study. During the study, two minipigs at a time were placed in slings in a calm and light-dimmed procedure room. The animals were lightly sedated with low-dose midazolam (0.3-0.7 mg/kg—increased during the five days as necessary to keep each animal calm) and their eyes were covered to keep them calm. Thereafter a mask was placed over the snout and the mask was connected to a Pari Boy® classic nebulizer. The nebulizer chamber was initially filled with 4 mL IS or saline, and was continuously refilled (three times @ 2 mL) until 10 mL was administered after approximately 30 min. According to the manufacturer, the residual volume is approximately 1.2 mL, therefore the administered dose was ~8.8 mL. A pulse oximeter was connected to the tail of each animal to measure pulse and oxygen saturation; measurements, including counting of respiratory frequency, were noted after 5, 10, 15, and 20 minutes of inhalation. After the procedure, the animals were placed in a recovery box and observed until full recovery and thereafter guided back to their stall. The procedure was repeated daily for five days. On day five, the animals were euthanized after the last inhalation.

Every morning before and afternoon after the procedure, all animals were scored to assess general condition, appetite, behavior, coughing, lung function, and mobility.

Blood samples were drawn the first day before inhalation (baseline) and on the last day of inhalation after the inhalation. Standard biochemistry and hematology, including differential count, were performed.

Routine necropsy with special attention to the respiratory system was performed following euthanasia by an experienced veterinary pathologist to observe potential macroscopic signs of toxicity in situ. Lungs and mediastinal lymph nodes were weighed. Samples for histopathology were collected proximally (including the main bronchus) and distally from the right cranial and the left caudal lung lobes, from the trachea, carina, mediastinal lymph nodes, heart (right and left ventricular muscles), kidney, and liver. The nasal passages were collected for histopathology by using a standardized approach to investigate three nasal levels.

Lungs (including trachea, carina, bronchi, and bronchioles), lymph nodes (sub carinal), nasal passages (the squamous, transitional, respiratory, and olfactory epithelium covering the nasal opening, nasoturbinate, maxiloturbinate, vomernasal organ, ethmoturbinates and nasopharynx), liver, kidney, and heart were examined histologically.

On a few occasions, a few coughs or a sneeze were heard in relation to inhalation or after removal of the mask from the snout; this was noted for one animal in the control group, two animals in the 50 ppm group, and one animal in the 100 ppm group. This could likely be a reaction to the humid local environment the mask creates around the snout. Since the incidence was similar between the groups, it is not considered to be attributable to IS. Respiratory rate, pulse, and oxygen saturation were similar between groups. Animals recovered from the mild sedation in maximum 10 minutes following dosing.

No clinical signs were seen at the regular daily checks. Hematological and biochemical parameters' development from baseline (Day 1 before inhalation) to after the experiment (Day 5 after inhalation) was unremarkable for all groups. Creatine kinase elevations which were seen in all groups are considered most likely due to struggling in relation to handling and blood collection.

At necropsy, no apparent macroscopic signs of toxicity were observed.

Pathological findings related to drug exposure were not observed in trachea, carina-area, and lungs. All minor macroscopic and microscopic findings seen in trachea, carina-area, and lungs were considered either related to the daily sedation procedure, non-successful attempts to sample blood from the jugular vein, euthanasia, or were incidental findings. For example, focal infiltration of alveolar macrophages, sometimes associated with mineralization were seen in some animals of all groups and is reported as a common finding in Göttingen minipigs. It has also been reported that euthanasia by pentobarbital can induce lung tissue damage including congestion, oedema, hemorrhage, emphysema, and necrosis based on studies in rats, mice, rabbits, guinea pigs, sheep, non-human primates, dogs, and cats, and consistency appears across species.

Within the nose and nasopharynx, hyperemia, epithelial desquamation, loss of cilia, and lymphoid hyperplasia were seen within all three groups. The changes were most often seen in focal areas. For the nasopharynx more animals were registered with changes within both the 100 ppm and 50 ppm HOCl groups when compared to the saline group. However, the description of the lesions was similar across the groups and based on the low number of animals it was concluded that no clear difference can be seen between the three study groups. It should be noted that epithelial desquamation can be seen as an artifact of tissue sampling. It is concluded that mask inhalation of 100 and 50 ppm HOCl could not be associated with an increase in findings of significance compared with the saline control group.

In conclusion, findings were seen in all groups, including the saline controls, but there were none in the IS treated animals that were considered to be attributable to IS. Based on this study, the NOAEL for IS was 100 ppm (8.8 mL) for administration by mask.

### Example 9.1.3: In Vivo Multi-Dose Safety Study

The IS has been tested in an in vivo inhalation model in minipigs to assess the maximum tolerated dose to aid the selection of doses in the subsequent GLP study. Healthy minipigs (n=6; 3 groups of 1 male and 1 female per group) were treated daily for seven days with aerosol concentrations of 1.2, 2.3, or 5.4 μg/L (using 50, 100, or 200 ppm HOCl+0.25% acetic acid, pH 5.5, isotonic) by a mask covering the snout. Daily treatment duration was 60 minutes and each animal received respectively 19.9, 19.1 or 22.2 mL in the groups dosed with 50, 100 and 200 ppm of IS daily. Animals were euthanized on Day 8, following 7 days of inhalation of IS. During the study, clinical condition, body weight, food consumption, hematology (peripheral blood), blood chemistry, organ weights, macroscopic pathology and histopathology investigations were undertaken. HOCl concentrations, calculated from the achieved aerosol concentrations and nominal hypochlorous concentration of the formulations, were 99%, 92% and 110% of target for Groups 1, 2 and 3, respectively. There were no test item related effects on clinical condition, body weight, food consumption, hematology or blood chemistry parameters, or organ weights and there were no, item-related macroscopic pathology or histopathology findings. It was concluded that IS was well tolerated when administered to Göttingen minipigs via a face mask for 60 minutes per day for 7 consecutive days and that the 50, 100 or 200 ppm concentrations were considered appropriate for 60-minute daily exposures on longer term toxicity studies in Göttingen minipigs.

### Example 10: Cytotoxicity of Wound Irrigation Solution (WIS)

In vitro cytotoxicity of a Wound Irrigation Solution (WIS) at 200 ppm HOCl has been evaluated in two cytotoxicity studies. The objective of the studies was to determine whether WIS is toxic to cultured mammalian L929 cells in vitro. The tests comply with the methods described in ISO 10993-5 and the formulation of the test items were prepared in compliance with ISO 10993-12. The following subsections summarize the in vitro cytotoxicity studies.

### Example 10.1: In Vitro Cytotoxicity of WIS (200 ppm HOCl)

The test item WIS (SOF 0001/05-01), containing 0.25% acetic acid and 200 ppm HOCl, pH: 4.3, was examined to determine the potential cytotoxic activity on cultured mammalian cells (mouse fibroblasts). The test was performed in accordance with the US Pharmacopeia, Method <87> and the ISO 10993-5 guidelines.

A formulation of WIS (SOF 0001/05-01) was prepared with complete cell culture medium (Ham's F12 medium supplemented with 10% fetal bovine serum and 50 μg/mL gentamycin). A diluent ratio of 0.2 g test item/mL diluent medium was used. This formulation was tested undiluted as well as diluted 1 part formulation+3 parts fresh cell culture medium. Positive control (sodium lauryl sulphate (SLS), 0.2 mg/mL) and untreated control cultures (served also as negative control, treated with complete cell culture medium) were included in the study. Triplicate cell cultures were treated at each test point for 48 hours. The control treatments produced appropriate responses, demonstrating the correct functioning and sensitivity of the test system. The diluted formulation showed no toxicity (cytotoxicity grade 0 in all cases), while the undiluted formulation showed cytotoxicity (cytotoxicity grade 4 in all cases).

Under the test conditions of this study (prolonged exposure, 48 hours), undiluted WIS (0.25% acetic acid and 200 ppm hypochlorous acid, pH: 4.3), showed cytotoxic effects on cultured L929 cells. Based on these results, it is concluded that WIS 0.25% acetic acid and 200 ppm hypochlorous acid, pH: 4.3 did not pass the requirements of ISO 10993-5 and USP<87> as the cytotoxicity grade was >2. However, the diluted formulation of WIS (SOF 0001/05-01) showed no toxicity (cytotoxicity grade 0 in all cases).

### Example 10.2: In Vitro Cytotoxicity of WIS (200 & 448 ppm HOCl)

In this study, the in vitro cytotoxicity of the WIS (200 ppm HOCl, 0.25% acetic acid), SOF 003/53 (448 ppm HOCl, 1% acetic acid) and SOF 003/51 (200 ppm HOCl, 1% acetic acid) formulations were evaluated. The applied in vitro assays measure the release of lactate dehydrogenase (LDH) from ruptured cell membranes and the metabolic activity (MTT) in the cell line NCTC clone 929 (L-929) after exposure towards the formulations for 1, 4, 24, and 48 hours. The assays were performed according to the EUNCL SOP (EUNCL-GTA-03).

For all the tested formulations, no significant membrane rupture was measured at the tested concentrations (10-0.005%) and exposure periods (1, 4, 24, and 48 hours).

According to the guidelines in the ISO-10993-5 international standard, none of the WIS formulations caused a cytotoxic effect (i.e., more than 30% reduction in cell viability) in the NCTC clone 929 (L-929) cells at the two shortest exposure periods (1 and 4 hours).

After 24 and 48 hours of exposure, the WIS did not have a cytotoxic effect on the cells, (i.e., less than 30% reduction in cell viability) whereas the SOF 003/53 and SOF 003/51 formulations induced cytotoxicity at these timepoints (i.e., reduced the viability by 40-45% after 24 hours of exposure and by 55-60% after 48 hours respectively).

### Example 11: Genotoxicity of Inhalation Solution (IS)

GLP in vitro studies with IS have been performed at Charles River Laboratories, Hungary.

### Example 11.1: In Vitro Bacterial Reverse Mutation Assay

An inhalation solution of the invention was tested for potential mutagenic activity using the Bacterial Reverse Mutation Assay. The study was performed according to GLP.

The experiment was carried out using histidine-requiring auxotroph strains of Salmonella typhimurium (Salmonella typhimurium TA98, TA100, TA1535, and TA1537) and the tryptophan-requiring auxotroph strain of Escherichia coli (Escherichia coli WP2 uvrA) in the presence and absence of a post-mitochondrial supernatant (S9 fraction) prepared from the livers of phenobarbital/β-naphthoflavone-induced rats. The study included a Preliminary Compatibility Test and an Assay 1 (Plate Incorporation Method). The following concentrations were selected and provided by the Sponsor with appropriate documentation as follows: 50 ppm, 100 ppm, 200 ppm and 500 ppm, these are equivalent to 0.05, 0.1, 0.2 and 0.5 mg/mL. At the highest treatment volume (500 μL) these were equivalent to 25, 50, 100 and 250 μg/plate; these concentrations were used in Assay 1. Due to cytotoxicity, additional treatment plate concentrations were also used with lower treatment volumes per plate of the 50 ppm test item concentration: 0.3162, 1.0, 3.162 and 10 μg/plate using treatment volumes of the supplied material at 6.3 μL, 20 μL, 63.2 μL and 200 μL, respectively. The maximum test concentration was 250 μg and the minimum was 0.3162 μg test item/plate (a total of eight concentrations). Inhibitory, cytotoxic effect of the test item (absent/slight reduced background lawn development) was observed in all examined bacterial strains without metabolic activation at 250, 100 and 50 μg/plate concentrations, and with metabolic activation at 250 μg/plate concentration.

In the assay the number of revertant colonies did not show any biologically relevant increase compared to the solvent controls. There were no reproducible dose-related trends and there was no indication of any treatment-related effect.

The reported data of this mutagenicity assay show that under the experimental conditions applied the test item did not induce gene mutations by base pair changes or frameshifts in the genome of the strains used, and therefore in conclusion, IS had no mutagenic activity under the test conditions used in this study.

### Example 11.2: In Vitro Mammalian Cell Micronucleus Assay

An inhalation solution of the invention was tested in an in vitro micronucleus test using mouse lymphoma L5178Y TK+/−3.7.2 C cells. The study was performed according to GLP. Two assays were performed (Assay 1 and Assay 2). In both assays, a 3-hour treatment with metabolic activation (in the presence of S9-mix) and a 3-hour and 24-hour treatment without metabolic activation (in the absence of S9-mix) were performed. Sampling was performed 24 hours after the beginning of the treatment.

The examined concentrations of the test item in Assay 1 (with and without metabolic activation) were selected and provided by the Sponsor as follows: 50 ppm, 100 ppm, 200 ppm and 500 ppm, these are equivalent to 0.05, 0.1, 0.2 and 0.5 mg/mL considering the treatment value which was 1 mL as determined in OECD No. 487 guideline (10% (v/v) in the final treatment medium. In Assay 1, the study was terminated because excessive cytotoxicity of the test item was observed. The selected concentration intervals were not sufficiently refined to evaluate at least three test concentrations to meet the acceptability criteria (within the appropriate cytotoxicity range). Any result with a Relative Increase in Cell Count (RICC) of <~40% was not acceptable for the assay, the aim should be to have a cytotoxicity of approximately 40%-50% achieved in the assay to demonstrate the concentrations used were sufficient to meet the guideline criteria. Therefore, an additional experiment (Assay 2) was performed with modified and more closely spaced concentrations to give further information about the cytotoxic effects and to meet the regulatory acceptability criteria.

The examined concentrations of the test item in Assay 2 (with and without metabolic activation) were the same as in Assay 1, however, additional lower treatment concentrations were applied. Therefore, acceptable concentrations of 10, 5 and 2 μg/mL (a total of three) were chosen for evaluation in case of the short treatment with metabolic activation, and concentrations of 6, 2 and 1 μg/mL (a total of three) were chosen for evaluation in case of the short treatment without metabolic activation, and concentrations of 7, 6, 2 and 1 ppm (a total of four) were chosen for evaluation in case of the long treatment without metabolic activation. None of the treatment concentrations caused a biologically or statistically significant increase in the number of micronucleated cells when compared to the appropriate negative (vehicle) control value in the experiments with and without metabolic activation.

In conclusion, IS did not cause statistically or biologically significant reproducible increases in the frequency of micronucleated mouse lymphoma L5178Y TK+/−3.7.2 C cells in the performed experiments with and without metabolic activation. Therefore, IS was considered as not being genotoxic in this test system under the conditions of the study.

### Example 12: Other Toxicity Studies

### Example 12.1: In Vitro Lung Surfactant Functionality

An Inhalation Solution of the invention was tested in a simulated alveoli model to evaluate its' effect on lung surfactant function. Lung surfactant reduces lung surface tension, allowing normal expansion and contraction during respiration. Inhalation of aerosols that interfere with the lung surfactant may induce a toxic response.

The test method involved exposing a small volume of lung surfactant to nebulized IS during simulated breathing cycles while quantifying lung surfactant surface tension. Change in surface tension was evaluated.

### Method

A previously well-described constant flow through set-up of a constrained drop surfactometer was used to test the product's effect on lung surfactant function. This method has shown 100% sensitivity in detecting harmful substances when compared to in vivo studies.

A drop of lung surfactant (10 μg) was exposed to nebulized 500 ppm HOCl IS (5 mL over five minutes) during simulated breathing cycles of the lung surfactant (to mimic an alveoli). The surface tension was evaluated continuously by axisymmetric drop shake analysis to detect potential critically low surface tension (below 10 mN/m) as would induce atelectasis in vivo.

### Results

No inhibition of the lung surfactant function was measured when lung surfactant was exposed to nebulized inhalation product in the highest concentration (500 ppm HOCl+0.25% acetic acid, pH 5.5, isotonic). Similar results were obtained for 0.9% NaCl (control).

### Example 12.2: Ocular Irritation Test Using the Isolated Chicken Eye Method

Since the solution will be delivered to the mouth and nose, a study to investigate possible irritant properties to the eye was performed. A GLP study, Test for Ocular Irritation: Isolated Chicken Eye Method with Inhalation Solution (SIS) was performed according to the method described in guideline OECD 438. Four concentrations of IS were provided by the Sponsor with respectively 500, 200, 100 or 50 ppm hypochlorous acid (HOCl). The study was performed over 2 days and each day was referred to as an Experiment (i.e., Experiment 1 and Experiment 2). In each experiment, three eyes were treated with 30 μL of test item (500 ppm or 200 ppm in Experiment 1 and 100 ppm or 50 ppm in Experiment 2). In each experiment three positive control eyes were treated in a similar way with 30 μL of 5% (w/v) Benzalkonium chloride solution. The negative control eye was treated with 30 μL of physiological saline (0.9% (w/v) NaCl solution). Corneal thickness, corneal opacity and fluorescein retention were measured and any morphological effects (e.g., pitting or loosening of the epithelium) were evaluated.

The results from all eyes used in the study met the quality control standards. The negative control and positive control results were within the historical control data range the in experiment. Thus, the study was considered valid.

According to the guideline, the outcome of this study is that the test substance is allocated to one of three categories; either non-irritant or severe irritant or that there is a requirement for further information. Based on this in vitro eye irritation assay in isolated chicken eyes with different concentrations of Inhalation Solution (SIS), the 500 ppm, 200 ppm and 100 ppm test item concentrations were classified as needing further information. An in vivo study is indicated at these concentrations. The 50 ppm test item concentration was classified as non-irritant.

### Example 13: Antibacterial Tests with Inhalation Solution (IS)

An inhalation solution of the invention was tested in an antibacterial assay against planktonically-grown gram-positive (Staphylococcus aureus) and gram-negative (Pseudomonas aeruginosa) bacteria, and it showed efficient killing of both bacteria at concentrations of 10-25 ppm HOCl. The tests were performed at Costerton Biofilm Center, University of Copenhagen. The results of such tests are provided in Table 4 below.

TABLE 4
Overnight-grown S. aureus and P. aeruginosa were diluted in fresh growth
medium and grown further for two hours, Thereafter, they were treated for one hour
with IS containing different concentrations of HOCl or saline as control.

| | Test solution | | | | | | |
|---|---|---|---|---|---|---|---|
| | 0.9% | 10 ppm | 25 ppm | 50 ppm | 100 ppm | 200 ppm | 500 ppm |
| Bacteria | NaCl | HOCl IS | HOCl IS | HOCl IS | HOCl IS | HOCl IS | HOCl IS |
| S. aureus (G⁺) | + | 1-log fold | − | − | − | − | − |
| | | reduction | | | | | |
| P. aeruginosa (G⁻) | + | − | − | − | − | − | − |

Plus (+) indicates growth, minus (−) indicates no growth.

Results: As seen in Table 4 antibacterial effect was seen already at 10 ppm HOCl for P. aeruginosa, and neither of the bacteria grew at 25 ppm HOCl.

Summary: Inhalation solutions of the invention efficiently eradicates gram-positive (S. aureus) and gram-negative bacteria (P. aeruginosa) at HOCl concentrations of 25 ppm and 10 ppm, respectively, and above. Full effect for all bacteria was seen for IS 25 ppm HOCl. Based on these observations, it is evident that any potential bacterial contamination during the manufacturing of IS, will be eradicated immediately in the product by the broad-spectrum antimicrobial activity of HOCl. Thus, production of any endotoxins is highly unlikely since it takes bacteria to produce endotoxins.

### Example 14: Antiviral Activity According to the Standard EN 14476 of IS (Starting with 25 Ppm HOCl), Against Vaccinia Virus

EN 14476 for general virucidal activity is conducted on chemical disinfectants and antiseptics. This is a quantitative suspension test for the evaluation of virucidal activity in the medical area and is performed by an accredited laboratory.

TABLE 5
Antiviral activity according to the EN14476 standard for IS (50 ppm HOCl)
diluted to 80%, 50% and 10% solution, against vaccina virus.

| | | | | | | | | | >4 log₁₀ |
|---|---|---|---|---|---|---|---|---|---|
| | | Interfering | Level of | log₁₀TCID₅₀/mL after . . . min | | | | | reduction |
| Product | Concentration | substance | cytotoxicity | 0.25 | 1 | 2 | 30 | 60 | after . . . min |
| test | 80.0% | clean | 1.50 | n.d. | ≤1.50 ± 0.00 | ≤1.50 ± 0.00 | n.d. | n.d. | 1 |
| product | | conditions | | | | | | | (RF ≥ 5.25 ± 0.33) |
| test | 50.0% | clean | 1.50 | n.d. | ≤2.50 ± 0.00 | n.d. | n.d. | n.d. | 1 |
| product | | conditions | | | | | | | (RF ≥ 4.25 ± 0.33) |
| test | 10.0% | clean | 1.50 | n.d. | 6.38 ± 0.41 | n.d. | n.d. | n.d. | >1 |
| product | | conditions | | | | | | | (RF = 0.38 ± 0.53) |

n.d. = not done

Results for IS: The test product of IS, 50 ppm as 50% dilution, 25 ppm HOCl, was able to inactivate the vaccina virus after 1 minute of exposure time under clean conditions (see Table 5). Therefore, the activity was not measured after 30 or 60 minutes. The reduction factor was ≥4.25±0.33 (1 minute). According to the EN 14476 standard, products that have antiviral activity against the vaccinia virus are considered active against all enveloped viruses.

Results for Hand and Surface Disinfectants: EN tests according to the biocidal product regulations were also performed on hand disinfectant and surface disinfectant solutions (HOCl, 200±30 ppm, HAc 0.25%, pH 4.3). The results show antimicrobial efficiency against E. coli, fungi, yeast, and vaccina virus (data not shown).

Summary: All EN tests show a fast and effective inactivation of yeast, fungi, bacteria, and viruses from 1 min to 30 sec according to the standard for hand disinfectant and surface disinfectants. The results of IS were not significantly different from the hand disinfectant solutions and indicate similar disinfecting properties, also at 25 ppm HOCl.

### Example 15: Antimicrobial Effectiveness Testing

Challenge testing may be performed according to USP42-NF37 2S chapter<51> efficacy or Ph. Eur. 5.1.3 antimicrobial preservation. solution batches (pH 4.3, representative HOCl batches to IS were challenged with various microorganisms and below, testing according to USP42-NF37 2S chapter<51> is presented in Table 6 below:

TABLE 6
Antimicrobial effectiveness of representative wound irrigation
solution (122 ppm HOCl, 0.25% HAc, pH 4.3). The results are presented
as the Log₁₀ values of the CFU counts.

| | Reference | Reduc- | Reduc- | Reduc- | |
|---|---|---|---|---|---|
| Micro- | value | tion at | tion at | tion at | |
| organism | (log) | 0 hours | 14 days | 28 days | Conclusion |
| E. coli | 6.60 | 5.60 | 5.60 | 5.60 | Acceptable |
| P. | 6.71 | 5.71 | 5.71 | 5.71 | Acceptable |
| aeruginosa | | | | | |
| S. aureus | 6.61 | 5.61 | 5.61 | 5.61 | Acceptable |
| C. albicans | 5.72 | 4.72 | 4.72 | 4.72 | Acceptable |
| A. | 5.45 | 4.45 | 4.45 | 4.45 | Acceptable |
| brasiliensis | | | | | |

Results and Summary: The acceptance criteria for antimicrobial efficacy test as described in USP 42-NF37 2S chapter <51> were met for all test microorganisms both at day 14 and day 28. In addition, the lowest concentration of IS may be tested according to USP42-NF37 2S chapter <51> for antimicrobial effectiveness.

### Example 16: Minimum Inhibitory Concentration (MIC) Tests

Determination of minimal inhibitory concentration (MIC) against five pathogenic bacterial strains was carried out by broth microdilution (dilutions of the highest concentration of the test substance, HOCl solution, pH 4.3, 100 ppm HOCl+1% acetic acid and dilutions), representative to IS. Following incubation for 24 hours after the treatment in the microtiter tray, the optical density was measured to evaluate growth. Furthermore, the suspensions were plated on agar and controlled for growth the following day. All tests were performed at Biofilm Test Facility, University of Copenhagen, Faculty of Health and Medical Sciences, Department of Immunology and Microbiology.

TABLE 7
Minimal inhibition concentration (MIC) of HOCl +
Acetic acid (pH 4.3) against microorganisms.

| Strain | MIC of HOCl + Acetic acid (pH 4.3) |
|---|---|
| S. aureus | 25 ppm and 0.25% |
| E. faecium | 25 ppm and 0.25% |
| P. aeruginosa | 25 ppm and 0.25% |
| A. baumanii | 25 ppm and 0.25% |

Results: For all microorganisms tested the MIC was 25 ppm HOCl+0.25% acetic acid. The growth was determined by both optical density (plate reader) and by growth on Müeller Hinton agar plates.

### Conclusion on Microbiological Attributes of IS

Results of the antimicrobial tests carried out with the inhalation solution of the invention (IS) product, starting at 10 ppm HOCl, as well as other representative HOCl formulations, clearly support that the IS product has excellent antimicrobial activity, and thus we are confident that also IS should be delivered devoid of any microorganisms, as for the other, representative products. This is due to the broad-spectrum antimicrobial activity of HOCl in the products, both at pH 4.3 and pH 5.5 (SIS), the acid form of HOCl in the solution is heavily dominating (≥99.1%). This is also supported by the literature reporting on the antimicrobial activity of HOCl (the acid form) and that HOCl has been used as a preservative to inhibit microbial growth in various health care products (e.g., wound irrigation/rinse solutions) already approved and sold on the market. Therefore, the IS is produced in a non-aseptic, non-sterile facility.

1-20. (canceled)

21. An isotonic inhalation formulation comprising from about 50 ppm to about 500 ppm of HOCl and from about 0.08 wt % to about 0.5 wt % of a pharmaceutically acceptable acid having a pKa between about 3 and about 5 and a diluent, wherein the formulation has a pH of 4.3-6.0.

22. The isotonic inhalation formulation according to claim 21, wherein the pharmaceutically acceptable acid is acetic acid.

23. The isotonic inhalation formulation according to claim 21, wherein the diluent is water or saline.

24. The isotonic inhalation formulation according to claim 22, wherein the isotonic formulation comprises from 50 ppm to 500 ppm of HOCl and from 0.08 wt % to 0.5 wt % of acetic acid, wherein the formulation has a pH of 4.3-6.0.

25. The isotonic inhalation formulation according to claim 21, wherein the formulation comprises:
a. from 50 ppm to 500 ppm of HOCl,
b. about 0.25 wt % of acetic acid; and
c. about 0.9% NaCl and/or about 5% glucose,
wherein the formulation has a pH of about pH 5.3.

26. The isotonic inhalation formulation according to claim 21, wherein the formulation is prepared for administration orally or by nasal inhalation.

27. A method of treating an airway infection in a subject in need thereof, the method comprises administering an effective amount of an isotonic formulation comprising from about 50 ppm to about 500 ppm of HOCl and a pharmaceutically acceptable organic acid, and a diluent, wherein the formulation has a pH of 4.3-6.0.

28. The method according to claim 27, wherein the pharmaceutically acceptable organic acid is acetic acid.

29. The method according to claim 27, wherein the isotonic formulation comprises from about 10 ppm to about 500 ppm of HOCl and from about 0.08 wt % to about 0.5 wt % of acetic acid, wherein the formulation has a pH of 4.3-6.0.

30. The method according to claim 27, wherein the isotonic formulation comprises about 100 ppm of HOCl and from about 0.08 wt % to about 0.5 wt % of acetic acid and a diluent, wherein the formulation has a pH of 4.3-6.0.

31. The method according to claim 27, wherein the isotonic formulation comprises about 200 ppm of HOCl and from about 0.08 wt % to about 0.5 wt % of acetic acid and a diluent, wherein the formulation has a pH of 4.3-6.0.

32. The method according to claim 27, wherein the isotonic formulation comprises about 500 ppm of HOCl and from about 0.08 wt % to about 0.5 wt % of acetic acid and a diluent, wherein the formulation has a pH of 4.3-6.0.

33. The method according to claim 27, wherein the isotonic formulation comprises from about 10 ppm to about 500 ppm of HOCl and about 0.25 wt % of acetic acid and a diluent, wherein the formulation has a pH of 4.3-6.0.

34. The method according to claim 27, wherein the isotonic formulation comprises from about 10 ppm to about 500 ppm of HOCl and about 0.5 wt % of acetic acid and a diluent, wherein the formulation has a pH of 4.3-6.0.

35. The method according to claim 27, wherein the isotonic formulation comprises:
a. from 50 ppm to 500 of HOCl,
b. about 0.25 wt % of acetic acid; and
c. about 0.9 wt % NaCl and/or about 5 wt % glucose,
wherein the formulation has a pH of about pH 5.3.

36. The method according to claim 27, wherein the method comprises preparing the composition for administration using a device configured to release a suitable amount of the composition, the device comprising:
a. a first compartment comprising NaOCl or Ca(OCl)₂,
b. a second compartment comprising a pharmaceutically acceptable organic acid; and
c. optionally, a third compartment comprising water or saline.

37. The method according to claim 27, wherein the isotonic formulation is administered by inhalation.

38. The method according to claim 27, wherein the infection is a lung infection.

39. The method according to claim 38, wherein the lung infection is a bacterial lung infection.

40. The method according to claim 39, wherein the bacterial lung infection is caused by a pathogenic gram-positive, or a pathogenic gram-negative bacteria.

41. The method according to claim 27, wherein the infection is caused by S. aureus, P. aeruginosa, E. coli, E. faecium or A. baumannii.

42. The method according to claim 27, wherein the infection is a pathogenic fungi lung infection.

43. The method according to claim 42, wherein the pathogenic fungi lung infection is caused by C. albicans or A. brasiliensis.

44. The method according to claim 27, wherein the subject is a human patient suffering from ventilator associated pneumonia (VAP).
